(19) United States
(12) Patent Application Publication
(10) Pub. No.: US 2012/0149128 A1
(43) Pub. Date: Jun. 14, 2012

(54) ASSAYS AND ASSAY DEVICES

(76) Inventor: Victor Manneh, San Diego, CA (US)

(21) Appl. No.: 12/711,226

(22) Filed: Feb. 23, 2010

Related U.S. Application Data
(63) Continuation-in-part of application No. 12/710,292, filed on Feb. 22, 2010, now abandoned.
(60) Provisional application No. 61/154,593, filed on Feb. 23, 2009.
(60) Provisional application No. 61/304,686, filed on Feb. 15, 2010.

(51) Int. Cl. G01N 33/566; G01N 30/00

(52) U.S. Cl. 436/501; 422/69

(57) ABSTRACT

Methods and apparatus for conducting analyte assays, including multiplexed assays are described. Such methods include assays adapted for low volume assay devices in which assays can be performed using undiluted biological liquid samples by exchanging binding medium with detection medium, using layered labels, and/or using droplet based mixing in an assay device.

### RELATED APPLICATIONS

This application claims priority to Manneh, U.S. patent application Ser. No. 12/710,292 filed 22 Feb. 2010, and claims the benefit of Manneh, U.S. Prov Appl 61/154,593 filed 23 Feb. 2009, and Manneh, U.S. Prov Appl 61/304,686 filed 15 Feb. 2010, each of which is entitled Assays and Assay Devices, and each of which is incorporated herein by reference in its entirety.

### FIELD OF THE INVENTION

The present invention relates to biological and biochemical assays.

### BACKGROUND OF THE INVENTION

The following discussion is provided solely to assist the understanding of the reader, and does not constitute an admission that any of the information discussed or references cited constitute prior art to the present invention.

Many different types of assays used for detecting biological analytes have been developed and used. In many cases, in order to achieve acceptable precision and accuracy, heterogeneous assays have been used. Such assays involve one or more wash steps to wash away unbound label from bound label. For medical applications, assays of this type are often relatively complex and frequently unsuitable for home use or even point-of-care use, and are thus restricted to laboratory testing. This often prevents results from being available sufficiently quickly to assist in diagnosis and/or treatment selection.

Other assays are commonly referred to as homogeneous assays, which do not require such wash steps. A difficulty in applying homogeneous assays to medical testing is that biological fluids often contain substances that substantially interfere with the analyte binding or signal generation or detection. In some cases, assays are configured to use sample dilution in order to reduce the interference. However, such dilution adds complexity and handling steps, often making such assays unsuitable for home or point-of-care applications.

Further, in attempting to provide reliable point-of-care assays and associated devices, some devices have been constructed to allow assaying using small volumes, i.e., microfluidic devices, and a number of different microfluidic assay devices have been described. For example, Buechler, U.S. Patent Appl. Publ. 2005/0147531 A1 (and related patents) describes “assay device structures for a device where fluid flows from one region to another.” The device structure includes “one or more capillarity-inducing structures; where the capillarity-inducing structure induces capillary force along an axis that is essentially perpendicular to the axis along which capillary force [is] induced in another region of the device.”(Abstract.)

### SUMMARY OF THE INVENTION

This invention concerns analyte assays, e.g., biological assays such as protein assays. In many embodiments, these assays can be highly suitable for use in point-of-care applications, or even home monitoring applications, as well as in medical laboratory applications, research laboratory applications, environmental field test applications, and others. The assays are generally configured to provide rapid, yet simple, assays with high precision. Many embodiments of the methods are well suited to assaying biological fluid samples, without requiring time-consuming wash steps or sample dilution steps required in many current heterogeneous or homogenous assays respectively.

In certain configurations, the present invention accomplishes this by using proximity signal modulation along with a partial separation or solution exchange that exchanges binding solution with reading or signal detection solution. The result is an assay with many of the advantages of conventional homogeneous assays, while achieving the precision that would otherwise only be achievable with a heterogeneous assay. The present partial separation or displacement assay systems can even be configured to provide multiplexing.

Also in some configurations, assays of the present invention use layered labels and/or use a novel protein coating method and/or the assays are configured in a distinctive strip format. Using an appropriate arrangement, it has been found that advantageous assays can be configured in strip format as wet assays using small sample volumes. Particularly advantageous configurations utilize droplet-based fluid manipulation, e.g., using an electrowetting or magnetofluidic approach.

Thus, in a first aspect, the invention provides a set of assay reagents, that includes a first analyte-specific binding reagent that includes a first label, a second analyte-specific binding reagent which includes a second label, where the first and second labels interact to provide a signal indicative of that interaction (advantageously using proximity signal modulation), and a complex separation moiety that is a part of the first binding reagent or the second binding reagent.

In particular embodiments, a signal from the labels is only generated which the first and second label are in close proximity; the separation moiety is or includes a magnetic material (e.g., magnetic beads), a surface binding moiety (e.g., a specific binding moiety or non-specific binding moiety), or an electrically charged moiety.

In certain embodiments, the reagents are configured to provide multiplexed analyte detection, e.g., for 2, 3, 4, 5, 6, 7, 8, 9, or 10 different analytes, or even more, or for at least a number of analytes as just specified. In particular embodiments, a plurality of members of the reagent set include distinguishable coding moieties; the distinguishable coding moieties include fluorescent dyes having different fluorescent emission peaks; the distinguishable coding moieties include fluorescent dyes having different absorption peaks; the distinguishable coding moieties include dye moieties having different absorption peaks; the distinguishable coding moieties include different chemiluminescent compounds having different luminescent wavelengths; the distinguishable coding moieties include enzymes having different enzymatic activities; the distinguishable coding moieties include particles having distinguishable light scattering properties; the distinguishable coding moieties include energy transfer dyes with differing emission wavelengths; the distinguishable coding moieties are prepared as full-coated, e.g., layered labels.

In some embodiments, the first analyte-specific binding reagent includes a photosensitizer and/or the second analyte-specific binding reagent includes a chemiluminescent compound that reacts with a product of a photosensitizer, usually a photosensitizer in the first reagent; the first analyte-specific binding reagent includes a first fluorescent compound and/or the second analyte-specific binding reagent includes a second fluorescent compound that accepts energy from a first fluorescent compound, usually a first fluorescent compound in the first reagent; the first analyte-specific binding reagent includes a first enzyme and/or the second analyte-specific binding reagent includes a second enzyme which uses a product of a first enzyme as a substrate, usually a first enzyme in the first reagent.

In certain embodiments, the set of assay reagents also includes a signal enhancer; the set of assay reagents also includes a reading solution. In particular embodiments, the first and/or second analyte-specific binding reagent includes or links with a full-coated label, such as a layered label, e.g., a layered label including a plurality of chemiluminescent or fluorescent molecules; the layered label includes a solid phase core, e.g., with chemiluminescent or fluorescent molecules embedded in the solid phase core, attached on the surface of the solid phase core, distributed in the coating layers of the layered label, and/or attached on the surface of the outermost coating layer of the layered label; the layered label does not include a solid phase core; the layered label is as described elsewhere herein. Likewise in particular embodiments, the first and/or second analyte-specific binding reagent includes or links with a fully linked coating label, e.g., a reduced disulfide protein coated label.

A related aspect of the invention concerns an assay complex that includes a first analyte-specific binding moiety that includes a first label, a second analyte-specific binding moiety that includes a second label, where the first and second moieties interact to provide a signal indicative of that interaction (e.g., using proximity signal modulation), an analyte bound to the first moiety and the second moiety, and a separation moiety, where the separation moiety is a part of the first binding moiety or the second binding moiety.

In certain embodiments, the complex includes pairs of analyte-specific binding reagents as described for the first aspect above or embodiments thereof or otherwise described herein for the present invention.

Another related aspect of the invention concerns an assay kit that includes a first analyte-specific binding reagent that includes a first label, a second analyte-specific binding reagent that includes a second label, where the first and second labels interact to provide a signal indicative of that interaction (e.g., using proximity signal modulation), a separation moiety, where the separation moiety is attached to the first reagent or the second reagent, and instructions for performing an analyte assay using the first and second reagents or the reagents are packaged together, e.g., in pre-measured quantities, or the reagents are both pre-packaged together and instructions are included in the kit.

In certain embodiments, the reagents are as described for the first aspect above or embodiments thereof or otherwise described herein for the present invention; the kit also includes a reading solution; the reagents are packaged in a single-use assay device, e.g., a microfluidic device; a plurality of such single-use assay devices are included in the kit, e.g., a plurality of microfluidic assay devices.

Yet another related aspect concerns a single-use assay device that includes a sample reservoir, a first analyte-specific binding reagent that includes a first label, a second analyte-specific binding reagent that includes a second label, where the first and second labels interact to provide a signal indicative of that interaction (e.g., using proximity signal modulation) and where the first or second binding reagent includes a separation moiety, a signal detection chamber in fluid connection with the sample reservoir, and a signal detection solution reservoir, where the first and second reagents are in fluid connection with the sample reservoir and the signal detection solution is in fluid connection wth the signal detection chamber.

In advantageous embodiments, the device is a microfluidic device; the device includes a plurality of coding labels providing distinguishably different detectable coding signals, where co-occurrence of a particular coding signal with a signal from the interaction of the first and second labels is indicative of the binding of a particular analyte; the first and second analyte-specific binding reagents are as described for the first aspect above or embodiments thereof or otherwise described herein for the present invention.

A further aspect concerns an assay reading device configured for reading assay results from assay devices configured according to the present invention. The assay reading device includes a separation controller (e.g., a magnetic and/or electric field controller configured to apply a magnetic field and/or electric field to an assay device positioned for reading in said assay reading device and thereby immobilize, retard, or move particles within the detection device that include a magnetic moiety and/or electric field responsive moiety), and at least one signal detector configured to detect signals indicative of analyte binding in the assay device for at least two different analytes.

In particular embodiments, the signal detectors include at least one photodetector (e.g., to detect light absorption or emission such as fluorescence or luminescence); the device includes at least two signal detectors.

In certain embodiments, the assay device is a described for the preceding aspect; the assay device is a home-use device or a point-of-care device.

The invention also involves methods for conducting assays. Thus, another aspect concerns a method for analyzing one or more analytes in a solution by forming an assay complex in a binding medium, displacing that binding medium with a reading solution, and detecting a signal from that assay complex.

In certain embodiments, the displacing is performed as a single-step displacement, e.g., a low volume or microfluidic displacement, e.g., utilizing no more than 10, 20, 30, 40, 50, 60, 70, 80, 90, 100, 120, 150, or 200 microliters.

In some embodiments, the one or more analytes is at least 2, 3, 4, 5, 6, 7, 8, 9, 10, or more analytes, or is specifically one of those numbers of analytes.

In certain embodiments, the assay complex includes a signal modulation label and a detection label.

In certain embodiments, the assay complex includes first and second analyte-specific binding reagents as described for the first aspect above or embodiments thereof or otherwise described herein for the present invention.

Likewise in another aspect relating to assays, the invention provides a method for enhancing detection of one or more analytes in a solution by retarding (which may be immobilizing) an analyte-specific sandwich binding complex in a flow device, displacing binding medium surrounding that complex by flow of a liquid reading solution, and detecting a signal indicative of the presence of the analyte from the binding complex in the reading solution, where the specific detection of the analyte is enhanced compared to detection in the binding medium; often the assay is a homogeneous assay; the assay utilized a proximity label.

In certain embodiments, the binding medium is blood, serum, plasma, urine, saliva, exhaled breath condensate, cerebral spinal fluid (CSF), vaginal fluid, male seminal fluid, or crude cell extract, and is diluted no more than 50 percent, e.g., diluted 0 percent, or no more than 5, 10, 15, 20, 25, 30, 40, or 50 percent or is in a range defined by taking any two of the values stated as endpoints of the range.

In certain embodiments, displacing is performed in a single step; the displacing is performed using no more than 10, 20, 30, 40, 50, 60, 70, 80, 90, 100, 120, 150, or 200 microliters of reading solution; the reading solution is selected to have lower quenching for singlet oxygen than the binding medium; the reading solution is selected to have lower fluorescent quenching than the binding medium; the reading solution is selected to have lower light scattering than the binding medium; the displacing involves no more than a 0.5, 0.6, 0.7, 0.8, 0.9, 1.0, 1.1, 1.2, 1.3, 1.5, 1.7, or 2.0× fluid volume exchange of the binding medium in the region from which signal is to be read.

In certain embodiments, the detecting involves detecting a plurality of signals indicative of the presence of a plurality of different analytes, e.g., at least 2, 3, 4, 5, 6, 7, 8, 9, 10, or more analytes, or is specifically one of those numbers of analytes.

In particular embodiments, the enhancing is conducted for an assay as described for the preceding aspect and/or using first and second analyte-specific binding reagents as described for the first aspect above or embodiments thereof or otherwise described herein for the present invention; the enhancing is performed using a kit and/or assay device as described herein for this invention.

Another aspect of the invention concerns a method for detecting the presence or amount or both of an analyte in a solution, and involves binding an analyte-specific binding construct with an analyte in a solution, and detecting a signal from a full-coat label (i.e., a layered label or staged label) linked with that analyte-specific binding construct, where detection of the signal is indicative of the presence or amount or both of the analyte in the solution.

In particular embodiments, the solution is blood (diluted or undiluted), plasma (diluted or undiluted), urine, exhaled breath condensate, saliva, cerebral spinal fluid (CSF), vaginal fluid, nipple aspirate fluid, male seminal fluid, crude cell extract solution, cell suspension, partially purified cells extract solution; such solutions may be from a mammal, e.g., a human, bovine, porcine, or ovine.

In certain advantageous embodiments, the solution is applied to a lateral flow assay device and the detecting is performed on that device, e.g., a lateral flow device as described herein or as described in any of U.S. Pat. Nos. 6,352,862, 7,238,537, 5,272,785, 5,602,040, 5,656,503, 5,622,871, 6,228,660, 6,156,271, 6,187,598, 7,109,042, 6,818,455, 5,714,389, 6,485,982, 5,989,921, 5,998,221, 5,182,216, 4,956,302, 6,130,100, or in any of U.S. Pat. Nos. 5,798,273, 6,136,610, 6,368,876, 7,132,078, 7,256,053, or 5,766,961 (each of which is incorporated herein be reference in its entirety); the signal is a colorimetric signal, a fluorescent signal which may be a time resolved fluorescent signal (TRF), a bioluminescent signal, a chemiluminescent signal, a radioactive signal.

For particular embodiments, the layered label includes a solid phase core bearing a plurality of detectable signal moieties and at least two (e.g., 2, 3, 4, 5, or even more) linked hydrophilic polymer layers coating the core; the layered label includes at least two (e.g., 2, 3, 4, 5, or even more) linked hydrophilic polymer layers comprising a plurality of detectable signal moieties embedded in the layers; the layered label includes a plurality (e.g., 2, 3, 4, 5, or even more) of linked hydrophilic polymer layers without a solid phase core; the layered label includes a solid phase core and at least two hydrophilic polymer coating layers, wherein the layered label has substantially less non-specific protein binding for proteins in undiluted human plasma than a coated label having the same solid phase core and a single coating of the same hydrophilic polymer as forms the outermost coating layer of the coated polymer.

In other embodiments, the label is a fully linked coating label, e.g., a label particle coated with reduced disulfide protein (such as reduced disulfide bovine serum albumin (BSA)). Such proteins are linked to particles (e.g., beads) under conditions such that at least one type of reactive groups, e.g., amines, are fully, substantially, or predominantly depleted. The beads may include or carry one or more detectable moieties (e.g., colorimetric or fluorescent dyes). The proteins preferably include one or more disulfide bonds, which upon reduction provide —SH groups availabel for reaction with other moieties. Such other moieties may, for example, be additional protein molecules which may be the same or different, or specific binding moieties, or signal generating moieties.

In particular embodiments, the label is a colorimetric label, a fluorescent label, a luminescent label, or a radioactive label.

A related aspect concerns a full-coat label, such as a layered particulate label which includes a plurality of polymer layers, where at least the outermost of those layers provides low non-specific protein binding, and a plurality of detectable label moieties.

In particular embodiments, the layered label includes 2, 3, 4, or 5 layers (e.g., polymer and/or protein layers) or at least 2, 3, 4, or 5 layers (e.g., polymer and/or protein layers; one or more outer polymer layers are permeable to water; the label includes a solid phase core, which may include a plurality of detectable signal moieties; the label lacks a solid phase core; for either a label with or without a solid phase core the layers, e.g., polymer and/or protein layers, include a plurality of detectable signal moieties; for either a label with or without a solid phase core a plurality of detectable signal moieties are embedded in the polymer and/or protein layers.

In certain embodiments, the label includes a plurality of binding moieties (e.g., avidin or streptavidin) which bind with an analyte-specific binding moiety (e.g., a biotinylated anti-analyte antibody); the label is linked with at least one analyte-specific binding moiety; the label is linked with at least one analyte-specific binding moiety and is linked with at least one analyte; the label is immobilized in a signal detection zone of a lateral flow assay device by linkage with immobilized analyte.

Likewise, in certain embodiments, the full-coat label is a fully linked coating label which includes a solid phase core coated with a highly linked protein coating, preferably substantially maximally linked.

In particular embodiments, the protein coating is BSA; the protein coating is linked to the solid phase surface through naturally occurring amine groups; a protein coating linked to the solid phase surface through amine groups has additional functional groups created by reduction of disulfide bonds in the protein to create —SH groups.

In particular embodiments, the label is a colorimetric label, a fluorescent label, a luminescent label, or a radioactive label.

Another related embodiment concerns an assay kit which includes a measured quantity of a first analyte-specific binding construct and at least one lateral flow assay device.

In advantageous embodiments, the assay device is configured to perform a wet assay; a wet assay device is configured to perform field mixing of sample and the first analyte specific binding construct in the device; the assay device is configured to assay a sample of 10 microliters or less; a controlled volume is extracted from a raw sample in the assay device; the mixing is performed using electrowetting effects; the analyte specific binding construct includes a detectable label; the detectable label is a colorimetric label, a fluorescent label (e.g., a TRF label), a luminescent label, or a radioactive label.

Another aspect concerns a method for detecting the presence or amount or both of an analyte in a solution, by depositing a fluid sample in a sample deposition zone of a lateral-flow assay device comprising a solid phase strip; depositing a specific binding reagent in a reagent deposition zone of said assay device, where the sample deposition zone and the reagent deposition zone may be the same or different; mixing the sample and the specific binding reagent using a field mixer to form a sample-reagent mixture, whereby the reagent specifically binds with analyte if any in the sample; migrating the sample-reagent mixture along the device to a signal detection zone; and detecting signal in the signal detection zone as an indication of the presence or amount or both of analyte in the sample.

In certain embodiments, the method also includes preparing the sample within the device, e.g., by separating liquid from cells, for example, blood cells.

In particular embodiments, specific binding reagent is applied with the sample; specific binding reagent is applied separately from said sample; specific binding reagent is dried onto a portion of the strip upstream of the signal detection zone.

In advantageous embodiments, the device also includes an electrowetting fluid manipulation electrode array an electrode array is used to mix a volume of said sample and/or used to move a volume of the sample into contact with the solid phase strip.

In a further aspect, the invention concerns a lateral flow assay device which includes a sample deposition zone, a reagent deposition zone, a field mixing zone, a solid phase strip in contact with the field mixing zone and including a signal detection zone, and a fluid collection zone in contact with the solid phase strip distal to the field mixing zone and the signal detection zone.

In certain embodiments, the field mixing zone includes an electrowetting fluid manipulation electrode array; the electrode array is configured to also move a droplet of fluid; the device also includes a filter or binding moiety or both selected to retain cells present in a sample; the solid phase strip is or includes nitrocellulose.

Likewise, in certain embodiments, the signal detection zone includes immobilized analyte-specific binding moieties; the fluid collection zone comprise an absorbent material; the device provides useful results when used with a liquid sample of 20, 10, 7, 5, 4, or 3 microliters or less; a sample volume of no more than 10, 7, 5, 3, or 2 microliters is passed over the solid phase strip.

As used herein, the term “analyte-specific binding reagent” refers to a molecule or complex that specifically binds to desired analyte, and may also include moieties having other functions, such as labeling the molecule or complex.

The term “label” is used in a manner common for biological or biochemical assays, and refers to a moiety of a molecule or complex that is directly or indirectly detectable in a manner providing detection of the presence or amount of the label present. Examples include fluorophores, chemiluminescent moieties, light absorbing moieties, resonance light scattering particles, enzymes, and the like.

The phrase “labels interact to provide a signal indicative of said interaction” and similar terms indicate that there is a transfer between two or more labels such that a signal can be detected that differs in level and/or type from a signal (if any) present in the absence of the interaction between the labels. The transfer between labels may be of various types, including, for example, chemical (such as singlet oxygen diffusing to a chemiluminescer, or one enzyme label producing a substrate for another enzyme), or energy (such as energy transfer between fluorescent labels). In most cases, the presence of the interaction signal functions as a proximity label indicating that the interacting labels are close together (the distance may depend on the characteristics of the labels).

The term “full-coated label” refers to a construct, usually a particle, which bears or includes detectable moieties and which is either a layered label as defined below or has at least one protein coating which is substantially fully linked to the surface below (i.e., a “fully linked coating label”), e.g., to the particle surface. In most cases, the protein will be fully linked through amines, e.g., such that accessible amines are substantially depleted. In most cases, such a full-coated label has one or more coatings which essentially fully cover the coated particle or interior portions of a layered construct which does not have a solid phase particle core.

The term “layered label” refers to a construct, usually a particle, which bears or includes detectable moieties and which has at least two layers of a polymer material. In many cases, there are covalent links between the layers. In most cases, the layers will be hydrophilic. The layered label may have a core particle, e.g., a polystyrene particle, or may be formed without a core. The detectable moieties may, for example, be covered by the layers, and/or may be distributed in or between layers. For layered labels having a core particle and detectable moieties covered by the layers, detectable moieties may be embedded in the core particles and/or on the surface of the core particles.

The term “staged label” refers to a construct, often a particle, which is protein coated with covalently linked protein. The protein is attached in a manner which essentially depletes functional groups of at least one type in the protein. Additional functional groups are then created in the protein, e.g., by reduction of disulfide bonds. Such constructs, e.g., particles, bear or include detectable moieties. The protein coating has one or more additional moieties linked through —SH groups resulting from reduction of the disulfide bonds or through functional groups derived from such reduced disulfide bonds. Such additional moieties may be of various types, for example, members of specific binding pairs (e.g., antigen for an antigen-antibody pair, or biotin for a streptavidin pair), detectable moieties, or additional coating species, which may be of the same or different protein or of a different type, e.g., a polysaccharide or synthetic polymer.

In reference to solution exchange, the term “displacement” or “displacing” refers to a limited volume solution exchange instead of a full wash, e.g., involving displacement of the prior solution (e.g., a binding solution) with a limited volume of displacement solution (e.g., a reading solution). The solution exchange will generally be limited to displacement of the prior solution with no more than about 2× the volume, commonly no more than about 2.5, 2.0, 1.7, 1.5, 1.4, 1.3, 1.2, 1.1, 1.0 or even less times the volume of the prior solution.

The terms “lateral flow assay” and “strip assay” are used herein equivalently to refer to assay formats, usually immunoassays, in which the test sample flows along a solid phase substrate (usually a membrane, which may be adhered to a backing material impervious to the liquid used in the assay) via capillary action from a sample application zone into a fluid sink. The sample encounters a detection reagent (commonly dried in a reagent pad downstream of the sample application zone; commonly a coloured reagent) which mixes with the sample and transits the solid phase substrate encountering one or more lines or zones which have been pretreated with an appropriate specific binding moiety (typically an antibody or antigen). Depending upon the analytes present in the sample the detection reagent can become bound at the test line or zone. After passing over the detection lines or zones, the fluid goes into a fluid sink (commonly an absorbent material).

In the present context, the term “separation moiety” or “complex separation moiety” refers to a portion or component of a molecule (e.g., a specific binding reagent)t that allows that molecule or a complex including such molecule to be immobilized or retarded (e.g., in a liquid flow) or moved. This allows, for example, displacement of the liquid around the molecule or complex can be by a new liquid. A particular example is a magnetic particle or material.

The term “wet assay” as used herein means an assay performed in or on a solid phase assay device in which reagents are added to the assay device in solution or suspension form as contrasted to a dry assay in which assay reagents are dried in the assay device, generally in an absorbent reagent pad. In most such dry assays, the sample is added in solution but binding and signal generation reagents are present in dry form and are reconstituted by the sample solution.

As used in connection with this invention, the term “field mixing” refers to mixing of fluids using varying electrical and/or magnetic fields, usually using droplets of the fluid. Similarly, the term “field fluid manipulation” refers to manipulation of a fluid using electrical and/or magnetic fields, e.g., mixing, movement, and/or droplet formation.

In connection with this invention, the term “fluid” refers to a liquid, e.g., an aqueous liquid.

Additional embodiments will be apparent from the Detailed Description and from the claims.

### BRIEF DESCRIPTION OF THE DRAWINGS

FIG. 1 schematically illustrates analyte-induced signal generation using proximity signal modulation label/detection label complexes.

FIG. 2 schematically illustrates the effect of using a reading solution to overcome solution-mediated signal interference.

FIG. 3 schematically illustrates the use of a set of different detection labels to identify different analytes in an assay based on proximity signal modulation label/detection label complexes.

FIG. 4 schematically illustrates the use of a set of different signal modulation labels to identify different analytes in an assay based on proximity signal modulation label/detection label complexes.

FIG. 5 schematically illustrates the use of one type of signal modulation label associated with distinguishably different coding labels to identify different analytes in an assay based on proximity signal modulation label/detection label complexes.

FIG. 6 schematically shows a complex similar to that shown in FIG. 5.

FIG. 7 shows a simplified assay device arrangement suitable for detecting binding complexes as in FIG. 6, in which coding labels and detection labels are detected sequentially at two different detection locations.

FIG. 8 shows the effect of exchanging binding medium with detection medium (reading solution) in removing interferents present in the binding medium in accordance with Example 1.

FIG. 9A shows a schematic configuration of a wet assay on a lateral flow assay device and FIG. 9B shows an exemplary calibration curve.

### DETAILED DESCRIPTION OF THE PREFERRED EMBODIMENTS

### A. Introduction

Many different assays have been developed for qualitative, semi-quantitative, or quantitative detection of the present of particular analytes, especially in solutions. In many cases, the analytes are present in biological samples, such as blood, serum, urine, and the like. A frequently complicating effect of such samples is the presence of a large number of other compounds, some of which can significantly interfere with detection of a particular desired analyte.

In many assays, the difficulties created by such interfering substances have been addressed by using heterogeneous assays, in which the target analyte is separated from the solution before detection. For example, often the target analyte is captured using a specific binding moiety attached to a solid phase substrate. The captured analyte is targeted by a label species that specifically binds to the captured analyte (either before or after capture). Unbound label species is washed away, and a detectable signal resulting from the presence of the label is detected, and is indicative of the presence and/or quantity of the analyte in the original sample.

Traditionally, such analyses were performed in specialized laboratories, and often required a day or even longer before results were reported. More recently there has been significant interest and development of assays that are simpler and/or faster to carry out. Desirably such simplicity and rapidity is not accomplished by sacrificing precision and accuracy. In some instances, these assays are homogeneous assays, in which the analyte is not separated from the original solution. The development of rapid, simple assays, including a number of homogeneous assay methods, has contributed to the development and use of point-of-use assays and devices for carrying out those assays. One advantage of such point-of-use assays and devices is that the assay results are generally availabel much more quickly, in some cases in a matter of minutes following initial sampling.

However, the presence of interfering substances in the samples has continued to be a limiting factor to the accuracy and precision of homogeneous assays, and especially of point-of-use homogeneous assays. In many cases, the problem has been addressed by using a significant level of dilution of the sample solution in order to reduce the concentration of the interfering substances. Unfortunately, this also reduces the signal strength and introduces an additional step that includes a need for a relatively large volume of dilution medium. For use in point-of-use or point-of-care devices, the dilution step introduces a potential source of error if performed manually before introduction of the sample into an assay device, or requires the assay device to be able to supply the relatively large dilution volume.

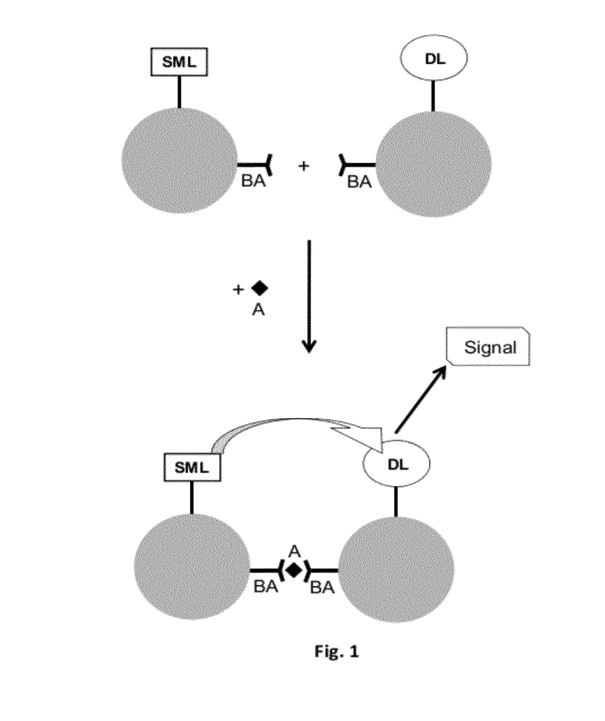

For some applications, the present invention addresses these difficulties in certain assay formats by providing a displacement method for at least partially purging interfering substances from around the analyte, along with proximity signal generation. Such proximity signal generation is shown schematically in FIG. 1. As illustrated, introduction of analyte (A) brings a binding agent (BA) labeled with a Signal Modulating Label (SML) into proximity with a BA labeled with a Detection Label (DL). The SML activates the DL producing signal. When not in proximity, the SML is incapable of activating the DL.

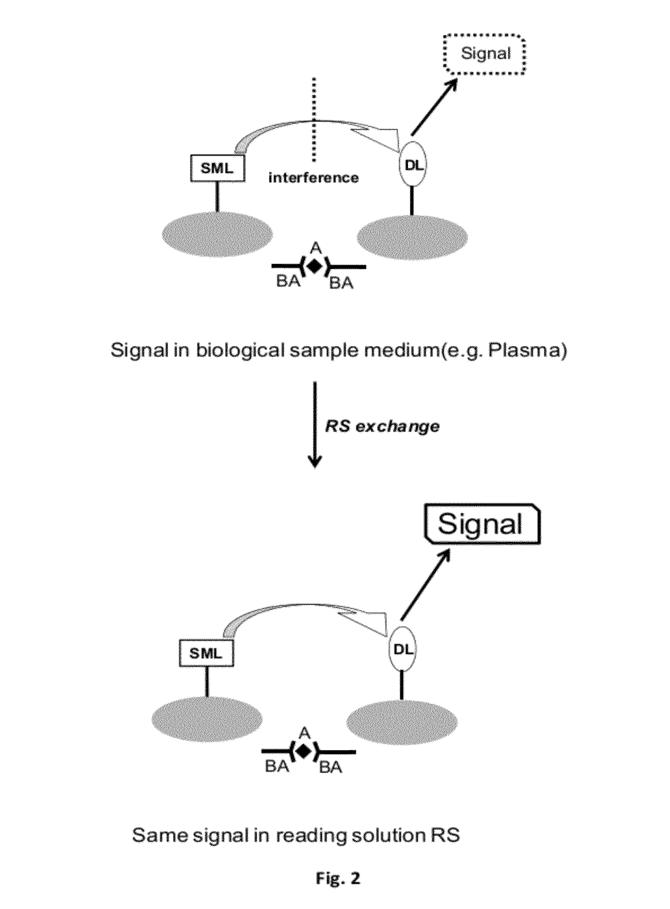

The effect of exchange of binding medium with reading solution (detection medium) is shown schematically in FIG. 2. Introduction of A brings a BA labeled with a Signal Modulating Label (SML) into proximity with a BA labeled with a Detection Label (DL). The SML activates the DL producing signal. The signal is quenched due to interferences in the biological sample. Introduction of reading solution regains the signal and removes interference. One exchange is sufficient to remove interferences due to biological sample. This method is readily adaptable to low volume assays, and in particular to point-of-use assay devices and methods.

### B. Homogeneous Assay Medium Displacement Method

As indicated above, the present methods utilize displacement of a binding medium by a detection medium (also referred to as a detection solution or reading solution or reading medium). Such displacement eliminates a large fraction of potential interfering substances which may be present in the sample and/or binding medium without the dilution step common or necessary in many homogeneous assay methods, and without the multiple wash steps used in many heterogeneous assay methods. In most cases, only a small volume of the detection medium is used to accomplish the displacement.

Typically, such displacement is accomplished by retarding, immobilizing, or otherwise controlling movement of the analyte complexes in a liquid flow and/or followed by a liquid flow. Following a volume of binding medium with a volume of detection medium in that flow displaces the binding medium surrounding the analyte complexes with a reading medium. In many cases, a simple 1× replacement of binding medium with detection (reading) medium is sufficient to substantially eliminate interference.

Advantageously, the detection medium can be selected to optimize the strength and/or consistency of the signal(s) to be detected. A number of ways in which such optimization can be accomplished are indicated below.

Such displacement can advantageously be carried out using very small volumes of medium, e.g., in small dimensioned flow channels. Assay devices adapted for the present assays can be integrated, such that they include all assay components except for sample. Such devices can also be constructed to retain all assay components within the device.

1. Retardation

A number of different techniques can be used to retard movement of assay complexes in the present methods. Of course, individuals familiar with assay construction will recognize the adaptations useful for applying the various retardation techniques. In cases in which such retardation is utilized, detection may be carried out with the complexes at a retardation (e.g., capture) location and still retarded, or may be released with detection performed with the complexes free in solution but still at or very near the retardation location, moved in solution to a new location (e.g., in a flow), or moved and recaptured subsequently in a different location for detection.

a. Magnetic Materials and Magnetic Fields

An advantageous retardation technique involves the use of magnetic materials in magnetic fields. In many cases, this is used in a flow environment, such that specific binding occurs in solution, the bound complexes are retarded (which can be immobilized) by creating a magnetic field which attracts the magnetic particles. This allows the binding medium to be replaced with another liquid, e.g., a reading medium.

A number of different magnetic materials can be used. Exemplary magnetic particles are described in Chandler et al., U.S. Pat. No. 6,773,812 B2, which is incorporated herein by reference in its entirety. The description indicates that “distinguishable subsets of microspheres can be constructed based on fluorescent intensities, and separations can be affected based on variable degree of magnetic content.” Magnetic bead materials are also described in U.S. Pat. Nos. 4,339,337, 4,774,265, and 5,356,713, all of which are incorporated herein in their entireties.

b. Charged Particles in Electric Fields

Similarly, instead of magnetic materials and magnetic fields, charged materials and electric field can be used to control movement of analyte/charged particle complexes. A variety of such charged particles are known and can be used in this invention.

c. Solid Phase Binding

Instead of the dynamic retardation processes using magnetic fields, electrical fields, or the like, binding to a solid phase medium can be used to immobilize the complexes. Such binding will, in many cases, involve specific affinity binding, e.g., with a member of a specific binding pair corresponding to a complementary member of that pair that is linked with bound analyte/label complexes, and not with unbound analyte.

d. Filtration

Instead of (or in addition to) solid phase binding, filtration can be used to immobilize complexes. Filters can, for example, be selected to provide suitable size cut-off, hydrophobicity or hydrophilicity characteristics, complex entrapment, and/or complex binding properties for a particular application.

2. Detection Solution

A variety of different detection solutions can be suitably used in the present assays and devices. The selected detection solution should be one in which the signal(s) from the labels in the assay can be effectively and consistently detected. In some cases, water, saline, or conventional buffers can be used, which do not themselves significantly interfere with the signal generation and/or detection.

In other cases, other solutions can be advantageous. For example, for assays which involve generation of singlet oxygen, it may be beneficial to use a solution which does not excessively destroy that single oxygen such that the singlet oxygen is unavailabel to the detection label (e.g., a chemiluminscer). While it is desirable for the singlet oxygen to be quenched sufficiently that the signal from the analyte binding complexes dominates the total signal, it is usually desirable for sufficient singlet oxyen to diffuse to the detection label or other label intended to be modulated by proximity of the singlet oxygen-producing species to provide a readily detectable signal. Therefore, various reading solutions can be selected with different quenching rates, e.g., based on the separation of labels in the analyte-bound complexes. Thus, for example, non-aqueous solutions (or solutions with non-aqueous co-solvent may be used in some cases.

Another type of reading solution may cause a change in the structure of composition of the complexes. For example, in cases where the moiety (e.g., particle) providing the ability to retard or move the complex interferes to an underirable extent with detection, a solution can be used which modifies the complex. Such modification can involve chemical modification of one or more moieties and/or separation or removal of one or more components of the complex. An exemplary method for removing one or more complex components is using cleavage of a disulfide bond (e.g., using DTT). Thus, if the linkage between a magnetic or charged particle and the respective label/analyte portions of the complex includes an accessible disulfide bond, that bond can be reduced and broken using DTT in the reading solution. After that bond is broken, the magnetic or charged particle and the label/analyte complex will separate. The separation can be increased by flowing the label/analyte complex away from the magnetic or charged particle, or by conversely by applying the magnetic or electrical field to move the magnetic or charged particle away from the label/analyte complex.

3. Low Volume Displacement

As indicated above, the displacement method is applicable to very low volume methods and devices. For example, in certain embodiments, the assay can be carried out with displacement using no more than 10, 20, 30, 40, 50, 60, 80, or 100 microliters. Such assays may, for example, be carried out in microfluidic assay devices.

### C. Exemplary Labeling and Detection Formats

The present methods can be carried out in many different ways, and can use many different materials and devices.

1. Luminescent Oxygen Channeling Assay (LOCI)

The present invention can advantageously be applied to a homogeneous assay method referred to as Luminescent Oxygen Channeling Assay (LOCI™ (Behring Diagnostics)), which is based on chemiluminescence. Generally, the assay generates a signal resulting from close approach of a photosensitizer moiety and a chemiluminescer. (See, e.g., Ullman, 1994, Proc Natl Acad Sci, USA 91:5426-30; Ullman et al., 1996, Clin Chem 42(9):1518-26; Ullman et al., U.S. Pat. No. 6,406,913, all of which are incorporated herein by reference in their entireties.) Upon irradiation with light, the photosensitizer produces singlet oxygen, which initiates luminescence from the chemiluminescer. Due to the dilution effect as the single oxygen diffuses away from the sensitizer and the short lifetime of the singlet oxygen, the effect is strongly dependent on the distance between the sensitizer and the chemiluminescer. Therefore, substantially all of the observed luminescence will be due to sensitizer/chemiluminescer pairs that are co-bound with an analyte.

2. Enzyme Multiplied Immunoassay EMIT

The EMIT assay is a homogeneous enzyme immunoassay (EIA). Typically the assay uses an excess of specific antibodies that bind with the analyte being measured, which are added to a liquid sample. If the target analyte is present, the analyte molecules bind to the antibody sites. Enzyme labeled analyte construct is added, where the binding of this construct to the antibody inhibits enzyme activity. The extent of binding of the construct to the antibody molecules will be inversely proportional to the concentration of analyte in the sample, and therefore will also be inversely proportion to the signal resulting from the activity of the free enzyme labeled analyte construct. That is, binding of the enzyme labeled analyte construct to the antibody binding sites not previously filled be sample analyte reduces the total signal from the enzyme. In most cases, the system produces a colorimetric signal.

3. Fluorescence Resonance Energy Transfer (FRET)

While Fluorescence Resonance Energy Transfer (FRET) may be used for generating the proximity signal, Time Resolved-Fluorescence Resonance Energy Transfer (TR-FRET) is said to be emerging as one of the preferred fluorescent assay formats in drug discovery laboratories. An example of such an assay is the LanthaScreen™ from Invitrogen.

The LanthaScreen™ format is based on the use of a long-lifetime terbium chelate as the donor species and fluorescein as the acceptor species. When terbium and fluorescein labeled molecules are brought into proximity, energy transfer takes place causing an increase in acceptor fluorescence and a decrease in donor fluorescence. These fluorescent signals can be read can be read in a time-resolved manner to reduce assay interference and increase data quality. The TR-FRET value is determined as a ratio of the FRET-specific signal measured with a 520 nm filter to that of the signal measured with a 495 nm filter, which is specific to terbium.

Other TR-FRET assays have used europium as the ‘long lifetime label’ and allophycocyanin (APC) as the donor species. Due to the large molecular mass of APC (>100 KD) it has typically been used as a streptavidin conjugate, to indirectly couple to the biotinylated substrate in a trimolecular FRET complex.

4. Fluorescence Polarization (FP)

Fluorescence polarization assays are based on the different polarization of the fluorescent output of bound versus unbound fluorescent species.

Fluorescence polarization (FP) is based on the observation that when a fluorescent molecule is excited by plane-polarized light, it emits polarized fluorescent light into a fixed plane if the molecules remain stationary between excitation and emission. Because the molecule rotates and tumbles in space, however, FP is not observed fully by an external detector. Light is differentially absorbed by molecules as a function of their orientation relative to the direction and polarization of the exciting light. The light subsequently emitted as fluorescence by each of the resulting electronically excited molecules will usually be polarized. Rotation during the lifetime of the excited states randomizes the orientation of the excited molecules leading to a net reduction in fluorescence polarization. The more rapid the tumbling the less polarization is observed. Fluorescence polarization immunoassay (FPIA), has been applied in the detection of analyte in a homogeneous assay format.

The FP of a molecule is proportional to the molecule's rotational relaxation time (the time it takes to rotate through an angle of 68.5°), which is related to the viscosity of the solvent, absolute temperature, molecular volume, and the gas constant. Therefore, if the viscosity and temperature are held constant, FP is directly proportional to the molecular volume, which is directly proportional to the molecular weight. FP of a large molecule is (with high molecular weight) is preserved because the molecule rotates and tumbles more slowly in space, while FP is largely lost (depolarized) for a small molecule (with low molecular weight), because small molecules rotates and tumbles faster. The FP phenomenon has been used to study protein-DNA and protein-protein interactions, DNA detection by strand displacement amplification, and in genotyping by hybridization.

This phenomenon was first applied in a homogeneous immunoassay by Dandliker (Dandliker, et al, 1961, 1973) but the method was initially little more than a laboratory curiosity because of the primitive state of development of commercial spectrofluorometers and the requirement for two separate measurements differing by a 90° rotation of a polarizing lens. Currently, over fifty fluorescence polarization immunoassays (FPIA) are commercially available, many of which are routinely used in clinical laboratories for the measurement of therapeutics, metabolites, and drugs of abuse in biological fluids.

Fluorescence polarization immunoassay (FPIA), has been applied almost exclusively to small molecule analytes. The sample and antibody are combined and the antigen in the sample competes with fluorescer-labeled antigen for binding to the antibody. Increasing concentrations of the antigen produce decreased polarization. Abbott Laboratories uses FPIA primarily for therapeutic drug monitoring and drug abuse testing on their TDx immunochemistry system. The success of the method after years of disuse stemmed in larger measure from the development of improved solid state methods for analyzing polarized light.

Interference from adventitious fluorophores and non-specific binding of the label to proteins as well as shielding the fluorescence signal by materials in biological samples has restricted the detection limit of FPIA to concentrations of above 100 pM. By using a highly hydrophilic long wavelength dye and time delayed measurements that permit discrimination between the emission from the background and the label, detection of concentrations down to 1 fM have been claimed (Devlin et al 1993). Exchanging the detection solution to maximize the efficiency of the signal would provide extremely advantageous environment to detect the unknown analyte at extremely low concentrations.

FP is expressed as the ratio of fluorescence detected in the vertical and horizontal axes and, therefore, is independent of the fluorescence intensity. This is a clear advantage over other fluorescence detection methods in that as long as the fluorescence is above detection limits of the instrument used, FP is a reliable measure.
Dandliker, W. B. & Feigen, G. Quantification of the antigen-antibody reaction by the polarization of fluorescence. Biochem. Biophys. Res. Commun. 5, 299 (1961).
Dandliker, W. B., Kelly, R. J., Dandliker, J., Farquhar, J. & Levin, J. Fluorescence polarization immunoassay. Theory and experimental method. J. Immunochemistry 10, 219-227 (1973).
Devlin, R., Studholme, R. M., Dandliker, W. B., Fahy, E., Blumeyer, K. & Ghosh, S. S. Homogeneous detection of nucleic acids by transient-state polarized fluorescence. Clinical Chemistry 39, 1939-1943 (erratum 2343), (1993).
Guo, X-Q., Castellano, F. N., Li, L., Lakowicz, J. R. Use of a long-lifetime Re(I) complex in fluorescence polarization immunoassays of high molecular weight analytes. Anal. Chem. 70, 632-637 (1998).

5. Enzyme Channeling

An assay format conceptually similar to LOCI is enzyme channeling. Enzyme channeling provides a method of detecting the proximity of two enzymes in an immune complex. The first enzyme catalyzes the formation of a substrate that is converted by the second enzyme into a detectable product. When both enzymes are independently dispersed in the same solution the rate of product formation is slow at first but accelerates as the concentration of the intermediate substrate builds up. This kinetic behavior changes when both enzymes are closely associated at a surface (Mosbach and Mattiasson, 1970). The local concentration of the intermediate in the vicinity of molecules of the first enzyme is determined by the rate of formation of the intermediate and its rate of diffusion away from the enzyme. A local steady state concentration is rapidly reached that is higher than the concentration in the bulk solution. Localization of several molecules of the first enzyme at a surface increases the rate of product formation and reduces the rate of product diffusion and thus increases its local concentration. When the second enzyme becomes bound to this surface it experiences a relatively constant elevated concentration of its substrate leading to a rapid linear rate of formation of the final product.

Homogeneous enzyme channeling immunoassays take advantage of this phenomenon (Litman, et al., 1980). Various surfaces have been employed including agarose particles, latex beads, and the polystyrene surface of a microtiter well. One enzyme serves to label an antibody or antigen and an excess of the other enzyme is bound to the surface. Usually the first enzyme is attached to the surface because more linear kinetics are obtained although channeling also occurs when the roles of the enzymes are reversed. A variety of enzyme pairs have been used including alkaline phosphatase/β-galactosidase, hexokinase/G6PDH, and glucose oxidase/HRP. When a natural substrate is not availabel as in the case of alkaline phosphatase/-galactosidase synthetic constructs can be prepared that permit the sequential reaction to occur.

A competitive assay for HIgG can be carried out with agarose particles labeled with HIgG and glucose oxidase (GO). Upon reaction with glucose these particles become surrounded by a halo of hydrogen peroxide. As the peroxide diffuses into the bulk solution it is diluted and the concentration is further reduced by catalase that is present in the reaction mixture. When HRP-labeled anti-HIgG antibodies bind to the particles in the presence of ABTS, an HRP substrate, there is a nearly constant rate of color formation that depends inversely on the concentration of the HIgG.

The most sensitive applications of enzyme channeling avoid the use of a pre-formed surface in favor of in situ formation of a colloidal precipitate. An assay for polyribose phosphate (PRP), a component of the cell wall of Haemophilis influenzae, was demonstrated using a reagent containing anti-PRP antibody labeled with GO (AB-GO), anti-PRP antibody labeled with HRP (Ab-HRP), and free GO (Ullman, et al, 1984). Combination of this reagent with a clinical sample to which anti-GO antibody had been added produced an Ab-GO:PRP:Ab-HRP sandwich complex that was incorporated into a colloidal GO:anti-GO immune complex (precipitin). Addition of glucose, ABTS, and catalase initiated the enzyme channeling reaction. The assay response was nearly linear with a detection limit of about 10 fM PRP in the assay mixture, sufficient for a cerebral spinal fluid assay for bacterial meningitis. Unfortunately there has been little study to determine if similarly sensitive homogeneous enzyme channeling immunoassays can be carried out using serum samples.
Litman, D. J., Hanlon, T. M. & Ullman, E. F. Enzyme channeling immunoassay: a new homogeneous enzyme immunoassay technique. Anal. Biochem. 106, 223-229 (1980).
Ullman, E. F., Gibbons, I., Weng, L., DiNello, R., Stiso, S. N. & Litman, D. Homogeneous immunoassays and immunometric assays. In Diagnostic Immunology: Technology Assessment and Quality Assurance. Eds. Rippey, J. H. & Nakamura, R. M., 31-46 (College of American Pathologists, Skokie, Ill., 1984).

6. Resonance Light Scattering

Yet another assay method is based on detection of light scattering from metal nanoparticles, e.g., gold particles. The use of such particles in assays is described in Yguerabide et al., U.S. Pat. Nos. 6,586,193 and 6,714,299 which are incorporated herein by reference in their entireties. (Also see, Yguerabide & Yguerabide, 1998, Anal Biochem 262:137-156; Yguerabide & Yguerabide, 1998, Anal Biochem 262:157-176, which are incorporated herein by reference in their entireties.)

### D. Multiplexing

Advantageously, a number of the present assay formats can be multiplexed, allowing detection of multiple analytes at the same time. Such multiplexing is of particular benefit in applications in which a panel of analytes is used as a diagnostic tool, e.g., for one or more diseases or conditions or for multiple drug detection assays.

The particular method by which multiplexing is accomplished will typically vary depending on the type of label used for detection. In order to perform multiplexed homogeneous assays, generally the signals produced are coded such that a particular signal or combination of signals corresponds to a particular analyte or set of analytes. In the simplest sense, distinguishable detectable signals are produced from differently labeled complexes corresponding to different analytes, i.e. g, different signal coding.

More particularly, in many cases the present assays involve the use of signal modulation labels where the signal modulation depends on proximity between a signal modulation label and a detectable label (which can include an energy transfer label which directly or indirectly transfers energy to a secondary detectable label). For the use of such labels, coding can be performed in a number of different ways.

For example, one way of accomplishing the multiplexing is to use a common signal modulation label, but to use distinguishably different detection labels. In this type of assay format, all of the complexes will include an analyte binding construct which includes the same signal modulation label (SML-BA). When the full complexes are formed (SML/BA-analyte-DL/BA) that signal modulation label modulates the signals from each of the distinguishably different detection labels in the complexes. Detecting the distinguishably different signals from the respective labels thus identifies the analyte bound in the particular complex. Examples of labels which are distinguishably different are chemiluminescent dyes and fluorescent dyes which emit light at distinguishably different peak wavelengths.

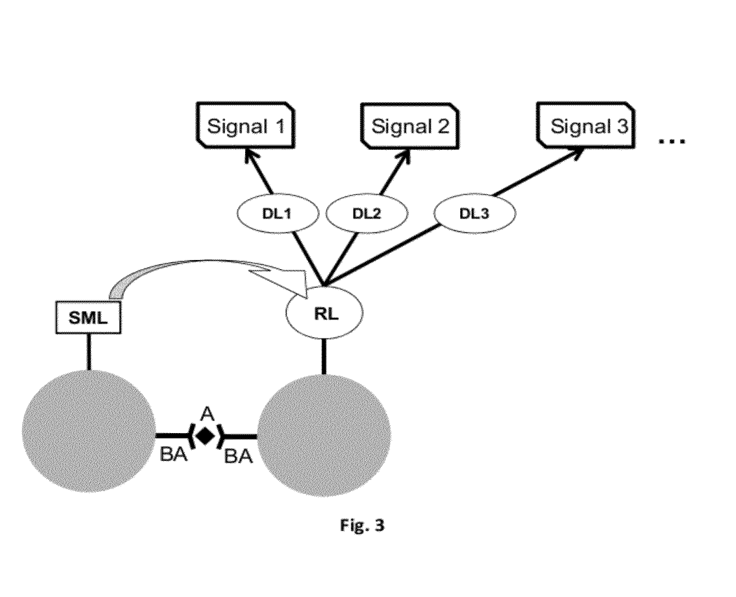

For example, the assay can be constructed such that distinguishable chemiluminescent moieties are used which correspond to different analytes. In this format, the different analytes can be distinguished based on the different luminescent signals resulting from formation of the sensitizer construct/analyte/chemiluminescer construct complex. This arrangement is shown schematically in FIG. 3. When brought into proximity (i.e. analyte induced binding), SML activates a Receiving Label (RL) that in turn activates multiple Detection Labels (DLs). Each DL produces a specific signal which codes for each analyte.

A second exemplary multiplexing technique applicable to such proximity signal modulation labels is to use detectably different signal modulation labels. Each of the set of signal modulation labels which can be distinguished, but each produces essentially the same modulation effect on the detection label(s). Usually, a single type of detection label will be used. The detection label signal thus identifies analyte-bound complexes, but does not distinguish between different analytes. The distinguishably different signal modulation labels then identify the particular analyte involved in each analyte-bound complex.

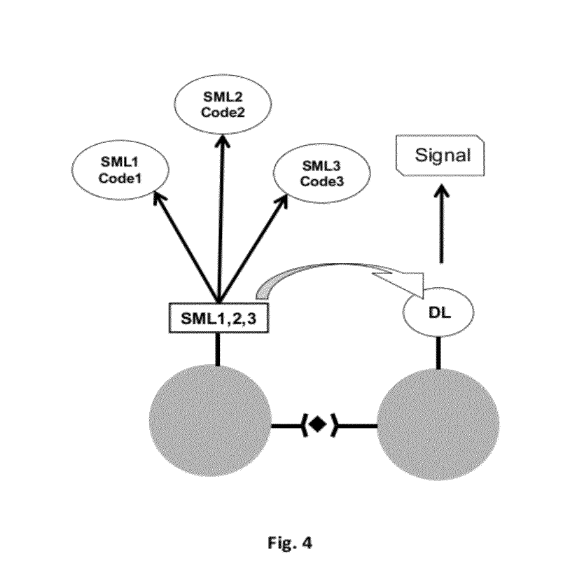

Thus, for example, a set of different sensitizer moieties can be used, which are distinguishably detectable. Different sensitizer moieties correspond to different analytes. The co-occurrence of the luminescent signal with the specific detection of the particular sensitizer therefore identifies the corresponding analyte. This arrangement is shown schematically in FIG. 4. When brought into proximity (i.e. analyte induced binding), various SML (SML1, 2, 3 . . . ) activate a DL producing signal. The various combinations of SML are distinguishable, each combination coding for a binding agent.

A third exemplary multiplexing technique applicable to proximity signal modulation labels involves the use of one type of detection label and one type of signal modulating label. Thus, the signal resulting from close proximity of the signal modulation label and the detection label identifies complexes that include bound analyte, but does not distinguish the different analytes. The multiplexing is achieved by using different codings for the signal modulation label construct and/or the detection label construct corresponding to the different analytes. Such codings can, for example, be provided by a separate coding moiety or moieties having distinguishable light absorbing or emitting properties.

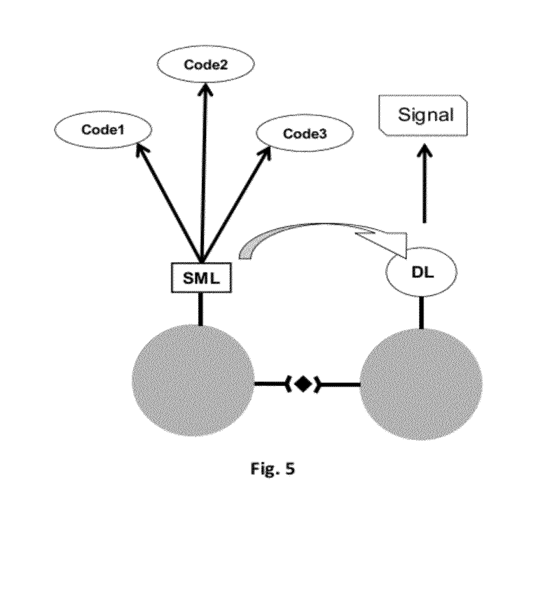

For example, this configuration can use a single type of chemiluminescer moiety, but the chemiluminescer construct has a different moiety which provides distinguishable detection of constructs targeted to different analytes. The coding may involve a single moiety, or may utilize multiple different moieties. For example, coding may be provided by using the co-occurrence of different combinations and/or ratios of different coding moieties. Similarly, distinguishable coding moieties may be provided with the chemiluminescer construct, either separately or in combination with coding on the sensitizer construct. This configuration is shown schematically in FIG. 5. When brought into proximity (i.e. analyte induced binding), SML activate a DL producing signal. The various combinations of SML are distinguishable, each combination coding for a binding agent.

Exemplary Multiplexing Approach

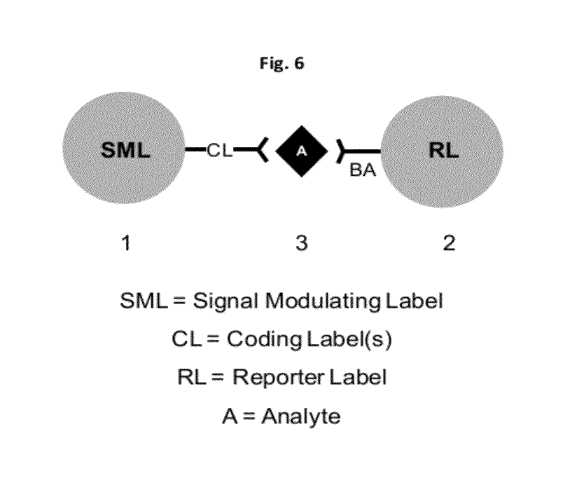

One of the formats for multiplexing homogeneous assays for proteins, nucleic acids, and other analytes is described utilizing set of coding molecule and a proximity signal modulating molecule on the same analyte-binding reagent to aid in measuring the concentration and detecting the identity of a corresponding set of analytes, any of which may be present in a sample. This configuration is illustrated in FIG. 6 (also see FIG. 5).

In FIG. 6, (1) is a binding reagent having a binding moiety, for example an antibody, that carries both coding labels (CL) to track the identity of the analyte and a signal modulating label (SML). (2) is a second binding molecule labeled with a reporter label (RL). When binding of an analyte (3) brings the SML and RL into proximity during a proximity binding assay, the RL produces a detectable signal. The detectable signal is independent of CML, CL or SML alone.

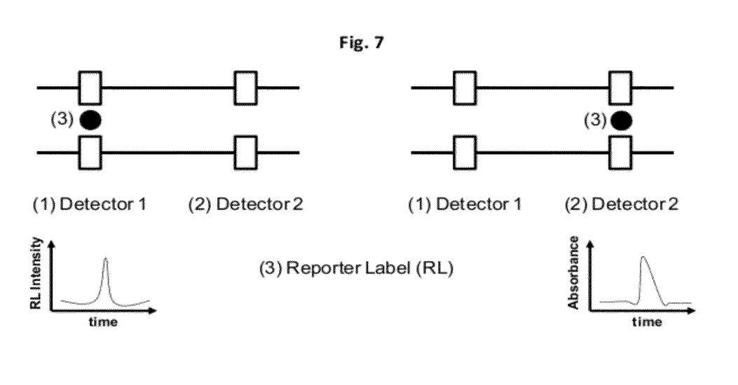

In FIG. 7, after the necessary reactions are complete, all of the components, i.e., coding labels, binding molecule, analyte, signal modulating label, and/or reporter label are transferred through a narrow channel for spectroscopic measurements. Included in the channel are two detectors—one designed for detection of the reporter label (1) and the other for detection of the coding labels (2). As a complex (3) passes by the signal intensity detector, signal intensity information generated by the reporter molecule is collected (see section on left). As a complex (3) passes by the coding label detector (2), analyte identification information is gathered from the coding labels (see section on right). The resulting information is combined to produce signal intensity on a per analyte basis that can be related directly to an analyte's concentration in the sample.

A specific example of a type of assay utilizing this homogenous-multiplexed scheme would use LOCI detection (see above description of technology), a separation step, and uniquely coded photosensitizer particles. In this assay, magnetic particles containing two different photosensitizing dyes (e.g. phthalocyanine and naphthalocyanine) are prepared at various dye loading ratios (i.e. 100% phthalocyanine (Pa), 100% naphthalocyanine (Na), 50%/50%, etc). Antibodies specific to antigens of interest are attached to the various magnetic-dyed particles, one type of antibody per magnetic-dyed particle type. Chemiluminescent particles with antibodies for each analyte are similarly prepared.

Upon introduction to a sample, formation of sandwich pairs of magnetic-dyed particles:antigen:chemiluminescent particles takes place. After separation of the magnetic particle complex from the sample, an optimized reading solution is introduced. The magnetic particle complexes are transferred to the narrow channel/detector for spectrophotometric measurement. Upon excitation of the photosensitizer dyes at the first detector, chemiluminescent signal is collected. As the complex continues down the channel to the second detector, its identity is determined from its spectral absorbance or fluorescent profile. Because the photosensitizer dyes have different spectral characteristics, resolution between the various magnetic-dyed particles, and, therefore, analyte that is bound, is possible. The resulting intensity measurements and coding information are combined to independently allow the determination of each analyte concentration in the same sample.

### E. Assay Devices

The invention also concerns assay devices adapted to carrying out the displacement method, as well as apparatus for reading and/or controlling the assay. Advantageously, such assay devices can be constructed to be suitable for point-of-use applications.

Also advantageously such assay devices can be microfluidic devices.

### F. Application to Cardiac Disease Marker Detection

As an example, the present invention can provide a new high sensitivity, nanoparticle based, homogeneous assay method with high quantum efficiency in biological samples. This method improves upon standard homogeneous binding assays, as described above, by removing interfering substances, thereby enhancing the quantum efficiency of the labels and improving the precision of the specific signal, allowing an assay platform of high sensitivity, speed, and simplicity to be developed for home use.

This assay platform can readily be applied to provide a home monitoring assay system, e.g., a Congestive Heart Failure (CHF) diagnostic that aids in stabilizing CHF patients at home. With over 5 million heart failure patients in the United States, and 550,000 new cases diagnosed annually, $33 billion is spent annually on the treatment and management of CHF. Many CHF patients showing symptoms of heart failure have their conditions spiral out of control and are rushed to the hospital. This diagnostic can be simple and precise enough to be used by patients themselves to establish an individualized baseline for the chronic monitoring of CHF, similar to the use of a glucometer for monitoring and stabilizing blood glucose by diabetics. Self monitoring and stabilization will significantly reduce the numbers of repeat hospitalizations that are common with CHF patients by allowing timely intervention prior to a serious cardiac event, thereby preventing the drama and expense of these far too common emergency room visits.

Furthermore, with the monitoring of additional markers, a point of care diagnostic for acute myocardial infarction (AMI) can be provided, to be used in such settings as Emergency Rooms and ambulances. This diagnostic product will quickly identify those patients in need of critical medical attention when therapy is most effective—immediately following a cardiac event. Such timely intervention can minimize cardiac damage, dramatically improving cardiac patients' prognoses.

Background and Significance

Heterogeneous and Homogeneous Assay Sensors

Many different assays have been developed for the qualitative, semi-quantitative, or quantitative detection of protein analytes, especially in solutions. In many cases, the analytes are present in biological samples (i.e. blood, serum, urine) or samples taken from similarly unknown and uncontrollabel environments (i.e. field samples, point of care samples). A frequently complicating effect of such samples is the presence of a large number of other compounds, some of which can significantly interfere with the detection of analytes (de Mello, 2003; Bjerner et al., 2002; Bjerner et al., 2004).

In most assays, the difficulties created by such interfering substances have been addressed by using heterogeneous assays, in which the target analyte is separated from the solution before detection. For example, often the target analyte is captured using a specific binding moiety attached to a solid phase substrate. The captured analyte is then targeted by a label species that specifically binds the captured analyte, and any remaining label that fails to bind is washed away. A detectable signal resulting from the presence of the label is collected and is indicative of the presence of the analyte in the original sample. A familiar example of a heterogeneous assay is the Enzyme-Linked Immunosorbant Assay (ELISA).

The need for complete label separation in heterogeneous assays is particularly problematic for use in non-laboratory environments. Typically in these environments it is desirable to use a disposable device that contains all of the reagents required for the assay of a single sample. Such systems have limited ability to store and/or use the large volumes of a wash buffer required for complete separation of the free and bound labeled assay components.

Homogeneous assay methods are ideal in such devices because the reagents are configured to produce a detectable change upon interaction with the sample (examples in Armenta et al., 1985, Blomburg et al., 1999, Engel et al., 1992, and Ullman et al., 1996). In homogeneous assays, the separation of the bound from the unbound label is avoided because the binding event modulates the signal from the label so that it is only necessary to measure the signal from the mixture of bound and free label.

However, the presence of interfering substances in the samples has continued to be a limiting factor to the accuracy and precision of homogenous assays, and especially of home care homogeneous assays. Examples of such substances include scattering materials (e.g. cells), antioxidants that interact with signal generation (i.e. vitamin C, glutathione), and absorbing materials (i.e. bilirubin, hemoglobin). In clinical settings, the problem has been addressed by using accurate pipetting of sample and a significant level of dilution in order to reduce the concentration of the interfering substances. This presents a problem because of the difficulty of quantitatively diluting the sample in an inexpensive home care assay unit and because of the reduction in sensitivity associated with significant sample dilution.

Technical Approach for Solving the Problem

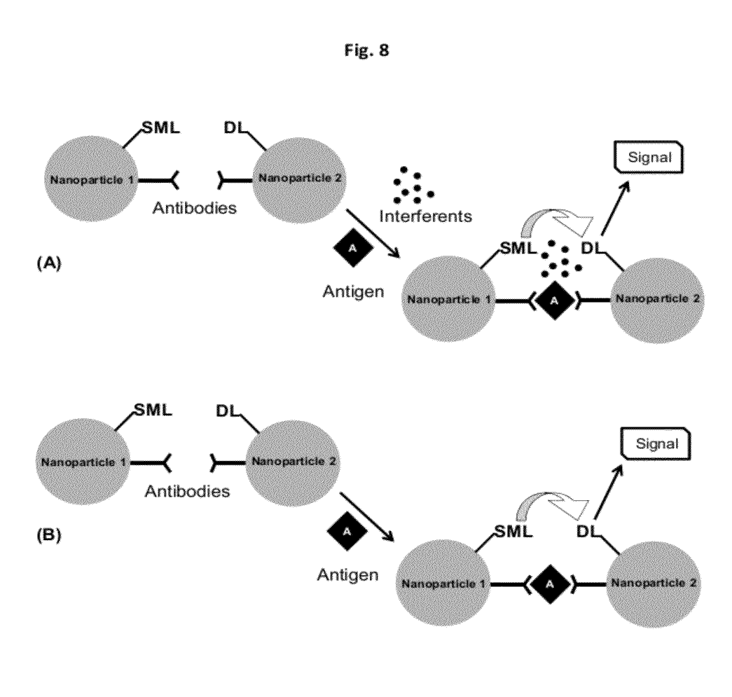

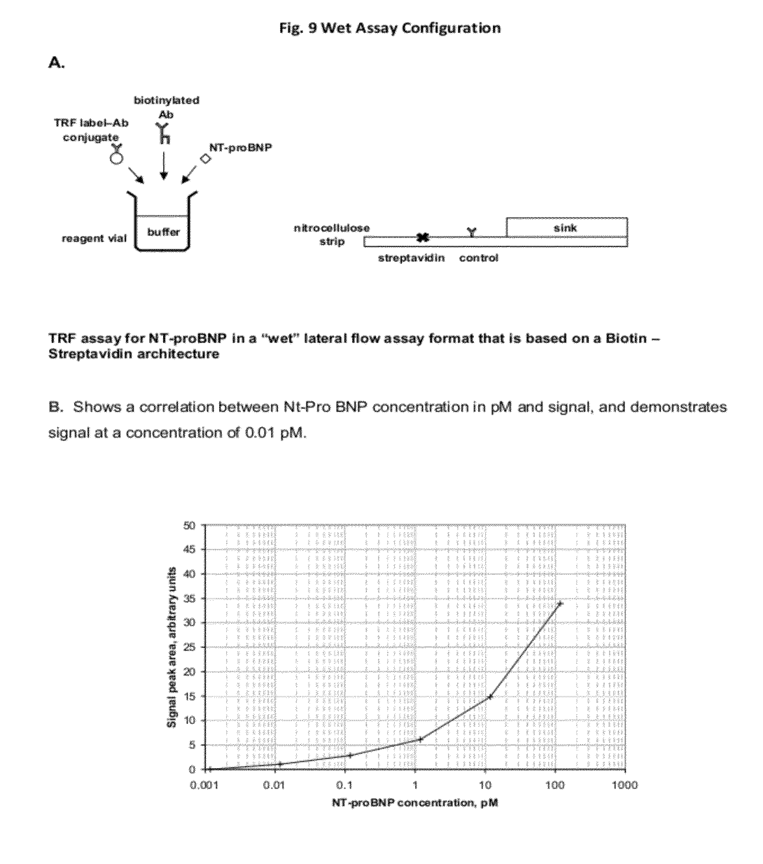

In accordance with the present invention, a highly sensitive, nanoparticle based, homogeneous assay method with high quantum efficiency in biological samples. This method improves upon standard homogeneous binding assays by removing interfering substances (but without the extensive wash steps required in conventional heterogeneous assay), thereby enhancing the quantum efficiency of the labels and improving the precision of the specific signal (see FIG. 9). Interfering substances are removed by trapping the substances and allowing this treated sample to incubate with the detection labels, then performing a limited separation or displacement step to remove unbound solutes from contact with detection labels. Failure to fully separate the bound and free labels does not affect the assay response because homogenous assays do not require separation. Introducing a reaction medium exchange adequately reduces the interfering substances so that assay sensitivity similar to that of standard heterogeneous assays can be realized without the requirement for multiple stringent washing steps used in conventional heterogeneous protocols. In many cases, a simple 1× or 2× replacement of sample medium with detection medium is sufficient to substantially eliminate interference (see FIG. 8 and Example 1).

Exemplary Applications of the Assay Technology

As indicated above, in advantageous embodiments the present technology can provide a home-care/point of care cardiac system. This system can be used to prevent frequent visits of CHF patients to the emergency room analogous to the use of a glucometer to prevent emergency events in diabetics. In constructing such a system and the associated reagents, the following may be used, though many alternatives and variations may also be used.
a) Reagents for Homogenous signal assay availabel for assay development.
   Dyeing of Latex Particles: Superparamagnetic nanoparticles with carboxyl functional groups (Bangs Laboratories) are stirred with 0.05 mg modified silicon Phthalocyanine dye (a photosensitizer) per one mg magnetic particle in 8:1:1 (vol/vol) ethylene glycol/benzyl alcohol/water for 8-10 min at 110° C. Following extensive washing to remove unincorporated dye, the absorbance of the particles is measured to determine the degree of dye incorporation. A similar procedure is followed for the dying of Acceptor particles using proprietary chemiluminescent dyes.
   Surface Modification of Latex Particles—Maleimide substituted amino-dextran are prepared by reacting amino-dextran (Invitrogen) with sulfo-SMCC followed by dialysis to remove unreacted SMCC. Typically, 35% of the amino groups are reacted to allow the rest of the amines to react with the surface of the dyed, carboxyl particles via EDC conjugation. After extensive washing, the maleimidodextran particles are incubated with excess thiolated Streptavidin (Prozyme) or thiolated, cardiac marker antibody (various antibody suppliers) for 16 h at 20° C. The remaining maleimide groups are capped with mercaptoacetic acid followed by excess iodoacetic acid to remove unreacted sulfydryl groups. Binding capacities are determined by incubation at 4° C. in assay buffer with periodic sonication containing either biotin-Flourescein for the streptavidin particles or ligand-Flourescein for the antibody particles and measuring fluorometrically.
   Quality of the reagents can be determined by their performance in a homogeneous assay as well as the nanoparticles' inherent characteristics (monodispersity, colloidal stability, ligand binding ability, antibody loading density, etc.)
b) Rapid assays for relevant cardiac markers developed
   Dose response curves for each marker obtained in both buffer and neat plasma samples. Sensitivity, Precision, Linearity, and Dynamic Range of each marker's assay is determined.
   Nearly equivalent signal intensities and precision should be generated from both sample types (buffer and biologic).
   Detection of 10 pM BNP, cTnl, and CKMB2 using homogenous assay reagents.
   Measurements to be performed on existing fluorometer as used in Preliminary Results section.
c) Reagents applied and response curves generated from a prototype detection chip.
   Detection chip (Cambridge Consultants) loaded into existing fluorometer for detection of developed assay.
   Similar dose response curves from the chip as seen previously should be observed.

Statistical Methods and Definitions

In evaluating a particular assay system, preferably all assay measurements are performed in triplicate (at minimum) with resulting mean and standard deviation from the mean calculated. Noise in an assay is defined as the standard deviation from the mean of a zero input (negative control). Assay sensitivity is defined as the input resulting in a signal-to-noise ratio of three. Precision information from all assays can be calculated by the percent coefficient of variation (% CV) defined as the standard deviation from mean/mean×100%. Assay Linearity can be calculated by the R² metric using the linear regression line through a plot of signal vs. input. Assay Dynamic Range can be calculated by dividing the highest input yielding a change in signal by the sensitivity of the assay.

Congestive Heart Failure (CHF) and Acute Myocardial Infarction (AMI)

CHF—CHF is characterized by left ventricular dysfunction resulting in reduced cardiac output. This in turn leads to a reduced exercise tolerance, poorer quality of life and a significant decrease in life expectancy. As a result of heart failure, the body initiates at least four compensatory mechanisms in an attempt to boost cardiac output. These mechanisms are mediated by the sympathetic nervous system and the renin-angiotensin system (Eichorn, 1998). These compensatory mechanisms result in cardiac hypertrophy (a consistent feature of CHF) that enables the heart to meet demands for increased cardiac output (Morgan, 1991) but is frequently associated with inter alia hypertension, aortic stenosis and myocardial infarction.

At a cellular level, the atrial natriuretic peptides (comprising a family of 25 peptides) are responsible for the regulation of extra-cellular fluid parameters within the heart including the volume and pressure of fluids within blood vessels. B-type or brain natriuretic peptide (BNP) was isolated, cloned and sequenced in 1998 (Sudoh et al., 1988). BNP is synthesized, primarily in the cardiac ventricle of humans, as an inactive pro-hormone precursor (proBNP) of 108 amino acids (αα), the appearance of which has been correlated with congestive heart failure. Processing of the proBNP results in generation of two peptides, N-terminal proBNP α α1-76 (NT-proBNP, the inactive cleavage product) and BNP α α77-108, the biologically active peptide, which has been found to be beneficial to the failing heart. This has stimulated commercial R&D of these two molecules as potential candidates as a prognostic indicator and a therapeutic product, respectively.

CHF patients are also prone to chest pain. Chest pain can be related to cardiac and non-cardiac events. Non-cardiac events can be as simple and benign as gastric discomfort or indigestion. Cardiac events, however, such as cardiac mediated arrhythmia, unstable angina and Acute Myocardial Infarction (AMI) can be life threatening.

Non-Q-wave AMI diagnosis relies heavily upon the detection of cardiac markers (Braunwald et al., 2000). During chest pain caused by a cardiac ischemic event such as unstable angina or myocardial infarction, damaged heart cells (myocytes) release their cellular content, including cellular proteins, into the blood stream. These proteins are used as markers to detect cardiovascular events and are collectively termed cardiac markers.

Currently, the diagnosis of AMI as defined by the World Health Organization (WHO) is based on the presence of two of the following three symptoms: ECG findings, chest pain symptoms and clinical history, and cardiac markers (Pedoe-Tunstall et al., 1994). However, current diagnostic criteria have been widely criticized because they do not always detect Non-Q-wave AMI, especially those patients with minor myocardial damage because:
The ECG does not provide a definitive diagnosis upon initial presentation in approximately 50% of patients (Apple et al., 1999).
A variety of diseases and disorders can cause chest pain, mimicking the symptoms of a heart attack. Thus, chest pain symptoms are nonspecific and, used alone, are not a reliable indicator of AMI (Schull et al., 2006).
Despite the widespread use of current cardiac markers, they are not sensitive enough to diagnose AMI in the first five critical hours after the onset of symptoms and frequently do not help the cardiologist (Morris et al., 2000).

Characteristics of Current Cardiac Markers

Current cardiac markers for evaluation of suspected AMI include b-type natriuretic peptide (BNP), Creatine Kinase-MB (CKMB), Myoglobin, CK isoforms, and Troponins (Jaffe, 2006). Recommended use for each marker varies according to its cardiac specificity, sensitivity, ease of measurement, turn around time for test results, and diagnostic and prognostic use. Many assays that measure the various cardiac markers are commercially availabel and their strengths and weaknesses vary are discussed (Wu, 1999). Therefore, selecting a cardiac marker that provides the most cost effective and clinically useful indicator of AMI is very difficult, and actually has not been found.

There is no single marker that satisfies all the requirements of the ideal marker and the panel of myoglobin, CKMB, and Troponin is insufficient (Apple et al., 1999). Troponin I, Troponin T and CKMB appear fairly late after the onset of chest pain. Myoglobin's level is elevated within two hours of myocardial injury and is a good tool in early diagnosis of AMI; however, Myoglobin lacks specificity for cardiac tissue and plasma elevation is seen with skeletal muscle injury, shock and renal failure. Some labs have chosen not to use Myoglobin testing because of such problems with specificity.

Creatine Kinase isoforms, CKMB2 and CKMB1, are good indicators of early stage infarction due to early and rapid release of CKMB2 from cardiac tissue into plasma and thus rapid and accurate diagnosis (Morris et al., 2000; Roberts, 1998). Creatine Kinase isoforms have high sensitivity for AMI and may cut CCU admissions by more than 50%. But the only test currently availabel is complicated and is difficult to perform, involving High Voltage electrophoretic separation, large instrumentation and highly trained personnel (Pentilla, 2002). It also measures CKMB activity, not mass, a value that is hard to correlate with the existing CKMB mass assay (measurement of total CKMB protein content) and it does not take into consideration loss of enzymatic activity during the high voltage electrophoretic separation.

In summary, rapid, differential diagnosis of chest pain for emergency physicians remains unmet. The current market situation for cardiac markers is described as follows:
There is no single, sensitive, and specific marker for myocardial infarction that rises within three hours after onset of symptoms and remains elevated for several hours.
There are no sensitive and specific laboratory tests either to diagnose perioperative myocardial infarction (PMI), reperfusion (a measure of whether treatment has been successful), or reinfarction, or to perform risk stratification of unstable angina patients.
The combination of Myoglobin, cardiac Troponin I (cTnl) and CKMB is less than ideal, because this triage does not provide specificity to the practicing cardiologist during the very early hours of an event when it is most needed.
High clinical demand exists for rapid, sensitive, and specific tests to diagnose AMI and to predict the risk of AMI in unstable angina patients.
Earlier diagnosis of AMI using biochemical markers may reduce mortality by enabling interventions such as thrombolytic therapy and angioplasty to be introduced sooner.
Ruling out AMI in the first few hours after patient presentation could reduce admissions to coronary care units (CCUs) by up to 70%, thereby saving billions in hospital costs. Conversely, better tests to “rule in” AMI will prevent the early release of high-risk patients from the emergency department.

An ideal marker combination should consist of a marker that rises rapidly (similar to Myoglobin) but is cardiac specific. The marker(s) should be sustainable and specific, and remain elevated for at least ten hours after AMI, thus providing a diagnostic time window, but a short enough period of time to preclude the detection of recurrent injury.

Assay Markers and Uses

BNP or NTpro BNP is produced by the ventricle when the ventricle cannot pump enough blood to meet the body's needs. Many factors, including age, gender, heart rate, obesity, renal function, and medications influence the circulating levels of BNP and NTpro BNP (Costello-Boerrigter, 2006). This makes it difficult to establish acceptable criteria for normal and abnormal levels, and reduces the utility of these novel biomarkers in diagnosing CHF.

This high variability in the BNP levels necessitates establishing individualized baseline levels per person with the patient monitored routinely. To accomplish this monitoring, a home test must be available.

Therefore, two major patterns will be followed by the patient and their physician:
1. Acute and dramatic increase of the BNP level, indicating an emergency situation and acute worsening in Congestive Heart Failure.
2. A chronic increase of the BNP levels, indicating an aggravation of the underlying disease state.

A high sensitivity cTnl assay coupled with a BNP assay offers an improved system for monitoring of CHF. This diagnostic will be simple and precise enough to be a point of use assay, used by patients themselves to determine a baseline for the chronic monitoring of CHF, similar to the use of a glucometer for diabetics' use in monitoring blood glucose. The BNP levels will be measured (normal level of BNP established for a particular patient) along with cTnl, which for a stable CHF patient should be very close to zero. Any worsening in the state of a CHF patient will manifest itself in an elevated level of BNP, and, combined with a spike in cTnl, would indicate a cardiac event is taking place with immediate medical care required. If BNP levels spike without an accompanying rise in cTnl this would indicate that other factors are in play and BNP levels could be returned to normal with diuretic use. The patient would continue monitoring BNP levels until a baseline level is again obtained.

For AMI, the same cTnl and BNP assays can be used along with a CKMB2 assay as an improved method for diagnosis. Indications of AMI would be an increase in cTnl, BNP, and CKMB2 levels. This assay and instrument will be a point of care instrument, used in such settings as emergency rooms and ambulances.

Although the initial system can be developed for markers in CHF and AMI, an extremely powerful system can incorporate additional markers to manage the continuum of Acute Coronary Syndrome (ACS). Markers can be identified and assays developed for the following conditions spanning the range of ACS progression:

Obesity

Diabetes,

Metabolic Syndrome,

Endothelial Dysfunction and

Plaque Build-up,

Inflammation

Plaque Rupture,

Thrombus formation

Ischaemia

AMI: Necrosis

CHF

Disease Management

In particular, any markers for plaque build-up and inflammation can be beneficially used because these are direct predisposing factors to the formation of the vulnerable plaque. Markers such as Oxidised LDL/B2GPI, Oxidised HDL, CRP, Cytokines, Plaque specific antigens, Adhesion Molecules (e.g. E-selectin), Glutathione, Lipoprotein A, Platelet activation factors, Urinary TxB₂ and Myleoperoxidase can be used in the present assays, enabling further monitoring of ACS.

### BACKGROUND LITERATURE

1. de Mello, A. J. et al. Dealing with real samples: sample pre-treatment in microfluidic systems. Lab Chip 3(1):11-19 (2003).
2. Bjerner, J., Nustad, K., Norum, L., Olsen K., and Bφrmer O. Testing and validating a homogeneous immunometric assay for interference. Clin. Chem. Lab Med. 42(2):208-14 (2004).
3. Bjerner, J., Nustad, K., Norum, L., Olsen K., and Bφrmer O. Immunometric Assay Interference Incidence and Prevention. Clin. Chem. 48:613-621 (2002).
4. Armenta, R., Tarnowski, T., Gibbons, I. & Ullman, E. F. Improved Sensitivity in Homogeneous Enzyme Immunoassays Using a Fluorogenic Macromolecular Substrate: An Assay for Serum Ferritin. Analytical Biochem. 146, 211-219 (1985).
5. Ullman, E. F. Recent Advances in Fluorescence Immunoassay Techniques. In Ligand Assay. Eds. Langan, J. & Clapp, 113-136 (J. Masson, New York, N.Y., 1981).
6. Farina, P. & Gohlke, J. R. Method for carrying out non-isotopic immunoassays, labeled analytes and kits for use in such assays. U.S. Pat. No. 4,378,428 (1983).
7. Blomberg, K., Hurskainen, P. & Hemmila, I. Terbium and Rhodamine as Labels in a Homogeneous Time-resolved Fluorometric Energy Transfer Assay of the -Subunit of Human Chorionic Gonadotropin in Serum. Clin. Chem. 45, 855-861 (1999).
8. Engel, W. D. & Khanna, P. L. CEDIA in vitro diagnostics with a novel homogeneous immunoassay technique. Current status and future prospects. J. Immunol. Methods 150, 99-102 (1992).
9. Ullman, E. F., Kirakossian, H., Switchenko, A. C., lshkanian, J., Ericson, M., Wartchow, C. A., Pirio, M., Pease, J., Irvin, B. R., et al. Luminescent oxygen channeling assay (LOCI™): sensitive, broadly applicable homogeneous immunoassay method. Clin. Chem. 42, 1518-1526 (1996).
10. Eichhorn, E. J. Restoring function in failing hearts: The effects of beta blockers. Am. J. of Med. 104(2), 163-169 (1998).
11. Morgan H E, Baker K M. Cardiac hypertrophy: mechanical, neural, and endocrine dependence. Circulation. 83:13-25 (1991).
12. Sudoh, T., Kangawa, K., Minamino, N., and Matsuo, H. 1988. A new natriuretic peptide in porcine brain. Nature. 332:78-81 (1998).
13. Braunwald, E, Antman, E M, Beasley, J W, Califf, R M, Cheitlin, M D, Hochman, J S, Jones, R H, Kereiakes, D, Kupersmith, J, Levin, T N, Pepine, C J, Schaeffer, J W, Smith, E E, Steward, D E, Theroux, P: ACC/AHA Guidelines for the Management of Patients with Unstable Angina and Non-ST-Segment Elevation Myocardial Infarction. JACC 36: 970-1062 (2000).
14. Pedoe-Tunstall H, Kuulasmaa K, Amouyel P, et al. Myocardial infarction and coronary deaths in the World Health Organization MONICA project. Circulation. 90: 583-612 (1994).
15. Apple, F S, Christenson, R H, Valdes, R, Andriak, A J, Berg, A, Duh, S-H, Feng, Y-J, Jortani, S A, Johnson, N A, Koplen, B, Mascotti, K, Wu, A H B: Simultaneous Rapid Measurement of Whole Blood Myoglobin, Creatine Kinase MB, and Cardiac Troponin I by the Triage Cardiac Panel for Detection of Myocardial Infarction. Clin Chem 45(2): 199-205 (1999).
16. Schull M, Vermeulen M, Stukel T. The risk of missed diagnosis of acute myocardial infarction associated with emergency department volume. Ann Emerg Med. 48 (6): 647-655 (2006).
17. Morris, S. et al., Clinical utility of CKMB isoform determinations in patients who present to the Emergency Department with continuous or resolved chest pain. J. Emerg Med. 19(1): 21-26 (2000).
18. Jaffe, A. Cardiovascular biomarkers: The state of the art in 2006. Clinica Chimica Acta, In-Press.
19. Wu, A. H. B. Cardiac Markers: From enzymes to Proteins, Diagnosis to Prognosis, Laboratory to Bedside. Ann Clin Lab Sci 29(1): 18-23 (1999).
20. Pentilla, K., et al. Myoglobin, creatine kinase MB isoforms and creatine kinase MB mass in early diagnosis of myocardial infarction in patients with acute chest pain. Clinical Biochem. 35 (8):647-653 (2002).
21. American Heart Association. Heart Disease and Stroke Statistics—2007 Update.
22. Gregory, D. et al. Hospital cost effect of a heart failure disease management program: The Specialized Primary and Networked Care in Heart Failure (SPAN-CHF) trial. Am. Heart Journal. 151(5):1013-1018 (2006).
23. Costello-Boerrigter, L., et al. Amino-Terminal Pro-B-Type Natriuretic Peptide and B-Type Natriuretic Peptide in the General Community—Determinants and Detection of Left Ventricular Dysfunction. J Am Col Cardiology. 47, 345-353 (2006).

### G. Coating to Reduce Non-Specific Binding and Layered Labels

In the conduct of many types of assays, non-specific binding is a major issue requiring resolution. Included in such situations is non-specific binding involving particular labels, such as enzymes, colored moieties, fluorescent particles, e.g., polystyrene particles bearing internal and/or external fluorescent moieties. In many cases, a coating is used, such as coating with BSA or with various synthetic polymers. However, in many cases, the single coating layers applied are inadequate so that appreciable and problematic non-specific binding still occurs.

Thus, the present invention also concerns labels which are coated in ways which advantageously reduce non-specific binding to assay surfaces and can also provide functional groups for attachment of additional moieties, e.g., full-coated labels, such as layered labels and/or as fully linked coating labels.

As indicated, some applications of the present invention utilize layered labels, which include multiple layers of coatings, e.g., 2, 3, 4, 5, or even more layers. An important application of such multiple layers of coatings is to reduce non-specific binding, but they can alternatively or in addition be used to carry detectable label moieties and/or other functional moieties. In most cases, the coatings, or at least the outer layers (e.g., outer two layers), are hydrophilic materials, typically hydrophilic polymers. The coating layers may be retained in place by interactions with the layer below and/or by intereactions within the particular layer. Such interactions include, for example, electrostatic interactions and covalent bonding. In advantageous embodiments, the coating is water permeable. Further, advantageous embodiments of such water permeable coatings have sufficiently open structure to allow access of water soluble molecules such as enzyme substrates, energy transfer dyes, and the like to penetrate below the top or outermost coating layer, and preferably even to layers lower than the second layer.

Thus, for example, a solid phase particle may be used as the core of the layered label which has detectable label moieties in and/or on the particle. The solid phase particle may be functionalized with a suitable reactive group (e.g., hydroxyl or carboxy) which can react with or be modified to react with functional groups in a first coating material. In many case, in order to functionalize the particle, it is first treated, e.g., by corona treatment, gas (e.g., air or oxygen) plasma treatment, flame plasma treatment, or chemical plasma treatment. Commonly such treatment introduces a functional group which may be used directly or used for attaching to or converting to a different functional group.

The first coating material includes either excess functional groups or another type of functional group which can be used to react with functional groups in a second coating. A similar process can be followed for additional coating layers. For example, alternating carboxy and amine functional groups may be used. In many cases, the outermost coating will be bound with a binding moiety, e.g., a member of a specific binding pair, such as one of a steptavidin (or avidin) biotin pair, or one of an antibody/antigen pair, or a receptor/ligand pair, or artificially derived specific binding pair. That binding moiety allows, for example, the particle to subsequently bind directly or indirectly with an analyte, such as in a sandwich arrangement.

For example, streptavidin may be attached to the coating. A biotinylated antibody binding to a particular cell surface antigen or other accessible moiety on the particle can then be used to link the layered label with the target particle, e.g., target cell. Of course, the system may be simplified, e.g., with an antibody attached to the coating, where that antibody binds to the target cell or other target particle. Spacers or linkers can also be included, e.g., to reduce steric hindrance to binding.

The layered labels can be configured in a number of additional ways. The layering may be formed on a core solid phase particle, e.g., a polystyrene particle, as mentioned above, where the particle bears detectable label moieties. Alternatively, the coating or moieties embedded in or attached to the coating may provide the detectable labels (alone or in conjunction with detectable labels directly associated with the core solid phase particle). Thus, for example, a layer may be coated over the solid phase particle, and detectable label moieties can be attached to or co-deposited with that coating. At least one additional coating may be laid over the first coating. Such additional coating can also have attached detectable labels, or the additional coating covers the label moieties attached to a lower coating layer or layers. Use of core solid phase particles usually provides a larger label, and may be useful, for example, to allow sufficient detectable label moieties to be present to provide desired signal intensity.

As another alternative, the core solid phase particle may be dispensed with, and a layered label may be created with multiple coating layers. In this case, the detectable label moieties are attached to or co-deposited with particular layers. One advantage of such a configuration is that the resulting layered label can contain multiple, even a large number, of individual label moieties. For example, if the label moiety is an enzyme, multiple enzyme molecules can be immobilized within the layered structure. Other types of label moieties can similarly be incorporated within the layered structure. As indicated previously, preferably the characteristics of the layer material (usually a hydrophilic material such as hydrophilic polymer such as a polydextran) allow fluid access to and/or detection of the internal label moieties.

As indicated, any of a variety of functional groups may be used for linking adjacent layers, for attaching detectable label moieties (e.g., dyes), and for attaching specific binding moieties (e.g., members of specific binding pairs). Important, commonly used groups for conjugation involve amine reactive, sulfhydryl reactive, carbohydrate reactive, carboxyl reactive, n-hydroxy succinamide active, photoreactive and/or ionic interactions. Exemplary groups include alcohol (e.g., as in ethanol), aldehyde (e.g., as in acetaldehyde), alkene (e.g., as in ethylene), alkyne (e.g., as in acetylene), amide (e.g., as in acetamide), primary amine (e.g., as in lysine), secondary amine (e.g., as in thymine), tertiary amine (e.g., as in triethylamine), carbonyl, carboxylic acid, disulfide, ester, ether, alkyl halide, ketone, nitrile, nitro, sulfide, thioester, thiol, epoxide, azide, N-hydroxy succinamide, anhydride, maleimide group, isothiocyanate, fluoroacyl imidazone, silane derivatives, silazane, and borate.

Linking functional groups can be used in connection with a large variety of polymers, including natural polymers, modified natural polymers, semi-synthetic polymers, and synthetic polymers. Examples of natural polymers which may be used for coating include complex mixtures such as serum, polypeptides (e.g., proteins such as BSA, casein, ovalbumin, lectins, or fibrinogen) and polysaccharides ((e.g., polysucrose, β-cyclodextrin-polysucrose polymer, dextrins (including both linear and cyclodextrins), dextrans (linear and branched), and chitin.

A number of synthetic polymers which may be used for coating, such as polyethylene glycol (PEG), polyvinyl chloride (PVC), polyvinyl alcohol (PVA), polyvinyl pyrrolidone (PVP), and non-ionic detergents such as Nonidet P-40 (NP-40) and Tween 20. Additional options are listed in the tables below.

| Name(s) | Formula | Monomer |
|---|---|---|
| Polyethylene | —(CH₂—CH₂)ₙ— | Ethylene |
| low density (LDPE) | | CH₂═CH₂ |
| Polyethylene | —(CH₂—CH₂)ₙ— | Ethylene |
| high density (HDPE) | | CH₂═CH₂ |
| Polypropylene | —[CH₂—CH(CH₃)]ₙ— | Propylene |
| (PP) different grades | | CH₂═CHCH₃ |
| Poly(vinyl chloride) | —(CH₂—CHCl)ₙ— | vinyl chloride |
| (PVC) | | CH₂═CHCl |
| Poly(vinylidene chloride) | —(CH₂—CCl₂)ₙ— | vinylidene chloride |
| (Saran A) | | CH₂═CCl₂ |
| Polystyrene | —[CH₂—CH(C₆H₅)]ₙ— | Styrene |
| (PS) | | CH₂═CHC₆H₅ |
| Polyacrylonitrile | —(CH₂—CHCN)ₙ— | acrylonitrile |
| (PAN, Orlon, Acrilan) | | CH₂═CHCN |
| Polytetrafluoroethylene | —(CF₂—CF₂)ₙ— | tetrafluoroethylene |
| (PTFE, Teflon) | | CF₂═CF₂ |
| Poly(methyl methacrylate) | —[CH₂—C(CH₃)CO₂CH₃]ₙ— | methyl methacrylate |
| (PMMA, Lucite, Plexiglas) | | CH₂═C(CH₃)CO₂CH₃ |
| Poly(vinyl acetate) | —(CH₂—CHOCOCH₃)ₙ— | vinyl acetate |
| (PVAc) | | CH₂═CHOCOCH₃ |
| cis-Polyisoprene natural rubber | —[CH₂—CH═C(CH₃)—CH₂]ₙ— | Isoprene |
| | | CH₂═CH—C(CH₃)═CH₂ |
| Polychloroprene (cis + trans) (Neoprene) | —[CH₂—CH═CCl—CH₂]ₙ— | Chloroprene |
| | | CH₂═CH—CCl═CH₂ |

| Monomer A | Monomer B |
|---|---|
| H₂C═CHCl | H₂C═CCl₂ |
| H₂C═CHC₆H₅ | H₂C═C—CH═CH₂ |
| H₂C═CHCN | H₂C═C—CH═CH₂ |
| H₂C═C(CH₃)₂ | H₂C═C—CH═CH₂ |
| F₂C═CF(CF₃) | H₂C═CHF |

| Formula | Type | Components |
|---|---|---|
| ~[CO(CH₂)₄CO—OCH₂CH₂O]ₙ~ | polyester | HO₂C—(CH₂)₄—CO₂H |
| | | HO—CH₂CH₂—OH |
| 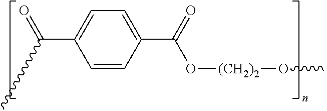 | polyester Dacron Mylar | Para HO₂C—C₆H₄—CO₂H HO—CH₂CH₂—OH |
| 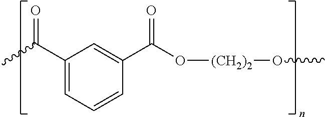 | polyester | Meta HO₂C—C₆H₄—CO₂H HO—CH₂CH₂—OH |
| 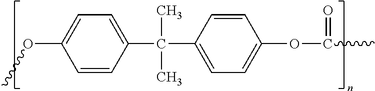 | polycarbonate Lexan | (HO—C₆H₄—)₂C(CH₃)₂ (Bisphenol A) X₂C═O (X = OCH₃ or Cl) |
| ~[CO(CH₂)₄CO—NH(CH₂)₆NH]ₙ~ | polyamide | HO₂C—(CH₂)₄—CO₂H |
| | Nylon 66 | H₂N—(CH₂)₆—NH₂ |
| ~[CO(CH₂)₅NH]ₙ~ | polyamide Nylon 6 Perlon | 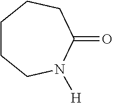 |
| 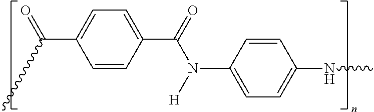 | polyamide Kevlar | Para HO₂C—C₆H₄—CO₂H para H₂N—C₆H₄—NH₂ |
| 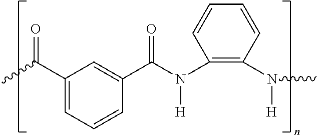 | polyamide Nomex | Meta HO₂C—C₆H₄—CO₂H meta H₂N—C₆H₄—NH₂ |
| 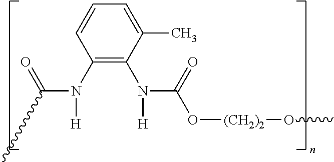 | polyurethane Spandex | HOCH₂CH₂OH 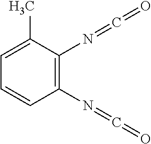 |

In addition to polymers formed of a single type of monomers, co-polymers can also be useful. As example of such a co-polymer is ABS rubber, which is a terpolymer of acrylonitrile, butadiene and styrene, and is commonly used for high-impact containers, pipes and gaskets.

Adjacent coating layers may formed of the same or different polymeric material depending on the desired properties. However, at least the outermost or two outermost layers should be selected to provide very low non-specific binding for proteins and/or other materials for which non-specific binding is undesirable in the particular assay.

In addition to layered labels, fully linked coating labels can be advantageous. Such labels differ from conventional coated labels in having a coating which is densely linked. For example, a protein such as BSA can be utilized and linked to a particle through accessible amine groups in the protein. When reacted at a high level the protein forms a substantially complete coating, and substantially all of the previously amine groups have been reacted. To provide new functional groups, e.g., for attachment of specific binding moieties, detectable label moieties, and/or other desired moieties, disulfide bonds can be reduced, making available —SH groups for such attachments.

Individuals familiar with this field will understand how to apply and link layers of various polymers using any of a number of different chemistries, and further how to attach binding moieties (e.g., a member of a specific binding pair, for example, streptavidin/biotin, antibody/antigen, or receptor/ligand (or ligand analog). A number of polymer materials suitable for this purpose are readily available.

### H. Assay Formats

The present invention also involves certain novel assay formats and associated reagents and kits. These assay formats can advantageously, but do not necessarily, include use of the full-coat labels such as layered labels or fully linked coating labels as described above. These assay formats are particularly advantageous for small laboratory, medical point of care, and/or home assays. In preferred cases, the assay is performed using a lateral flow assay device, preferably approved for use as a small laboratory, point of care, and/or home care diagnostic device. The assay devices can be configured as qualitative (presence/absence), semi-quantitative (above or below a threshold or within a specified range), or quantitative (distinguishing various levels of analyte in samples and optionally giving a numerical result) devices.

In one type of assay format, lateral flow assays (also referred to as strip assays) such as those described in Davis, Davis et al U.S. Pat. No. 4,889,816; Davis et al; Davis et al U.S. Pat. No. 7,238,537; May et al U.S. Pat. No. 5,275,785; May U.S. Pat. No. 5,602,040; May et al U.S. Pat. No. 5,656,503; May et al U.S. Pat. No. 5,622,871; May et al U.S. Pat. No. 6,228,660; May U.S. Pat. No. 6,156,271; May et al U.S. Pat. No. 6,187,598; May et al U.S. Pat. No. 7,109,042; May U.S. Pat. No. 6,818,455; Charlton et al U.S. Pat. No. 5,714,389; Charlton U.S. Pat. No. 6,485,982; Charlton et al U.S. Pat. No. 5,989,921; Charlton U.S. Pat. No. 5,786,228; Charlton U.S. Pat. No. 5,786,227; Charlton U.S. Pat. No. 5,981,293; Charlton et al U.S. Pat. No. 6,673,614; Jeng et al U.S. Pat. No. 5,064,541; Malick et al U.S. Pat. No. 5,998,221; Malick et al U.S. Pat. No. 6,194,220; Schuler et al U.S. Pat. No. 5,798,273; Clayton et al U.S. Pat. No. 5,182,216; Gordon et al U.S. Pat. No. 4,956,302; Jobling et al U.S. Pat. No. 6,130,100; Penfold et al U.S. Pat. No. 6,133,048; and Ching et al. U.S. Pat. No. 5,780,308, each of which is incorporated by reference in its entirety. A related assay and device is described in Allen, U.S. Pat. No. 5,580,794 which is incorporated herein by reference in its entirety.

In some applications of the present invention, assays and assay devices as described in the patents listed above are used with a label which is multiply layered as described above. In the above-listed patents, the assays generally utilize binding reagent immobilized in a reagent pad. Sample is applied upstream of the reagent pad, mobilizing the reagent and coating a strip in contact with but downstream of the reagent pad. Nitrocellulose is commonly used for the strip surface. The strip is also in contact with a fluid sink, so that the sample is drawn through the reagent pad, across the strip, and into the fluid sink. The reagent includes a binding reagent which binds to analyte in the sample. The strip includes a detection zone (i.e., signal detection zone) where analyte in the sample is immobilized, along with corresponding binding reagents from the reagent pad, generally in a sandwich arrangement. In the above-listed patents, the detectable labels are directly detectable, but indirectly detectable labels, such as fluorescent labels, can also be used.

Another set of strip assay formats that can utilize the present layered labels are wet assay formats. In such wet assay formats, the binding reagent is not dried in a reagent pad linked between a sample pad and a membrane strip as generally described for the patents listed above, but instead is added to the solid phase, either together with or separately from the liquid sample. Thus, for example, the sample and binding reagent can be mixed together and the mixture applied to the assay strip.

In another variant, the binding reagent in a lateral flow assay device is dried on the membrane rather than in a reagent pad) between a sample application zone and a signal detection zone.

In certain particularly advantageous formats, fluids in an assay device are physically manipulated by applied forces. Such manipulation is useful, for example, for mixing and/or for fluid movement and/or for droplet formation. For example, fluids can be manipulated using electrical or magnetic fields. Among other advantages, these approaches allow manipulation of fluids in an assay device with essentially no loss of fluid. This makes possible the construction of assay devices which utilize very small sample volumes, e.g., 1-5 microliters. Advantageous fluid manipulation techniques allow fluid manipulation in droplet form.

For example, such fluid manipulation can be performed using electrowetting effects, e.g., as described in Pamula et al., US Pat Appl Publ 2007/0045117, entitled Apparatuses for Mixing proplets; Pamula et al., U.S. Pat. No. 6,911,132, iss. Jun. 28, 2005, entitled Apparatus for Manipulating proplets by Electrowetting-Based Techniques; US Pat Appl Publ 2007/0037294, entitled Methods for Performing Microfluidic Sampling; US Pat Appl Publ 2007/02410, entitled proplet-Based Washing; US Pat Appl Publ 2007/0243634, entitled proplet-Based Surface Modification and Washing; and US Pat Appl Publ 2008/0105549, entitled Methods for Performing Microfluidic Sampling, each of which is incorporated herein by reference in its entirety for all purposes, specifically including for their descriptions of fluid mixing and other fluid manipulation using electric fields.

Thus, for example, the present devices can utilize a relatively simple version of the electrowetting electrode array and control circuitry to create, mix, and/or move droplets of sample material. In such devices, a liquid sample is applied upstream of a solid phase material which bears a signal detection zone. If the sample includes cells from which it is desirable to separate the liquid, the cells can be immobilized (e.g., using a binding to specific binding moieties such as antibodies) and a volume of the liquid moved away from the cells. A small volume of liquid from the sample can be mixed with binding reagent and/or other desired reagents in various ways, e.g., by pre-mixing reagent with sample, by passing the liquid through a reagent pad, or by passing liquid over or through a portion of the solid phase material on which the reagent has been dried. Advantageously the sample liquid encounters binding reagent within the region where the electrowetting fluid manipulation is performed. Following the last mixing, a small volume of liquid is moved into contact with the solid phase material. The liquid transits that material (usually by capillary action), encountering a line(s) or zone(s) where analyte becomes immobilized. Usually the line or zone is created by immobilizing a suitable member of a specific binding pair (e.g., an antibody) on the solid phase material. Liquid passes over the signal detection line or zone, typically into a fluid sink, which may include an absorbent material. In most cases, a detection moiety is used (e.g., conjugated with a specific binding moiety which links the detection moiety with analyte) which becomes immobilized with analyte at the signal detection line or zone. Detection of signal at that line or zone indicates the presence and/or amount of the particular analyte in the sample.

In some cases the strip assay device includes it own read-out. Thus, for example, the device may provide a direct detection label, such as a colorimetric label, or may include components to utilize an indirect detection label, such as a fluorescent label. Thus, in devices using a fluorescent labels, the device can include a light source (e.g., an LED) which emits light of a suitable wavelength to generate the fluorescent signal.

Similar to the electrowetting approach, a magnetofluidic approach may be used, e.g., as described in Garcia et al., US Pat Appl Publ 2008/0213853, or in Brauner et al., US Pat Appl Publ 2008/0220539, both of which are incorporated herein by reference in their entireties for all purposes, specifically including for description of fluid mixing or other fluid manipulation using magnetic fields.

### EXAMPLES

The following example illustrates a basic application of an aspect of the present invention, with analyte binding occurring in an undiluted liquid biological sample, and the replacement of the biological liquid with a reading solution utilizing magnetic immobilization of analyte/label complexes.

### Example 1

### Proximity Binding Assay in Undiluted Plasma Samples: Interference of Undiluted Plasma in Homogeneous Assay and Recovery of Signal

Experimental Protocol:

Materials—Superparamagnetic Particles (Bangs Laboratories) containing the signal modulating label (SML) phthalocyanine were prepared to create a Magnetic Particle containing singlet oxygen donor reagent—SML reagent (Mag-SML), were functionalized with streptavidin. Aldehyde functionalized particles (Perkin-Elmer) containing a dioxetene chemiluminescent dye and time-resolved fluorescent dye were functionalized with amino-PEG-biotin (Molecular Biosciences) via reductive amination with sodium cyanoborohydride (Sigma) to create detection labeled particles (DLP). Reactions were carried out under conventional conditions.

Experimental—Four reactions containing Mag-SML and DLP were incubated in Phosphate buffered saline, 0.1% Tween (PBST) buffer to form Mag-SML-DLP complexes. As a set of negative controls, four reactions containing Mag-SML, DLP plus excess free biotin (Sigma) were also incubated to prevent specific binding. Half of the reactions received buffer while the other half received neat human plasma. A magnetic field was applied to four of the reactions (two buffer and two plasma reactions) thereby holding the magnetic particles in place, and the initial medium (buffer in two of the reactions, plasma in the other two) was exchanged once with the detection solution PBST (Reading Solution) (See FIG. 1)

See FIG. 10 for a schematic pictorial description of the Experimental Set-up.

Signals from each reaction were quantified by exciting the signal modulating label with a 300 mW, 680 nm light source and collecting the resulting time resolved fluorescent (TRF) signal between 520 and 620 nm from the detection label using an instrument configured for such detection (Perkin-Elmer). The amount of signal collected was then compared to that obtained from the non-exchanged buffer sample (defined as 100% signal) to determine the degree of interference and/or signal recovery. Signals from the negative control reactions were examined to indicate the specificity of the binding reactions.

Results—quantitative results are shown the table below.

TABLE 1
Describes quantitative results and percent signal of
treatment from control for different assay conditions.
Control was run by performing the assay without
buffer exchange

| | | Counts per | % signal |
|---|---|---|---|
| Sample | Medium and Treatment | second | recovered |
| 1 | Buffer | 40572 | 100 |
| 2 | Plasma | 16225 | 40 |
| 3 | Buffer, 1X Buffer Exchange | 42834 | 106 |
| 4 | Plasma, 1X Buffer Exchange | 41490 | 102 |
| 5 | Buffer + 3 mM biotin | 42 | 0.10 |
| 6 | Plasma + 3 mM biotin | 348 | 0.86 |
| 7 | Buffer + 3 mM biotin, 1X Buffer | 101 | 0.25 |
| | Exchange | | |
| 8 | Plasma + 3 mM biotin, 1X Buffer | 244 | 0.60 |
| | Exchange | | |

Samples 1 and 5—Sample 1 demonstrates the power of signal produced due to Streptavidin-biotin specific binding and bringing the DLP in close proximity to MAG-SML to form the Mag-SML-DLP complex formation. Specific inhibition of complex formation was abolished by the addition of free biotin (sample 5). The same number of particles was present in both mixtures. This confirms that the signal generation mechanism is due to a proximity binding event and DLP must bind Mag-SML in order to generate signal.

Sample 2 exhibits the interference effect observed in undiluted plasma in a standard homogenous assay protocol. The signal intensity was reduced by 60%.

Sample 4 illustrates the power of the present homogenous assay protocol, because one exchange of the undiluted plasma sample with a reading solution (RS) results in complete signal recovery and enhance assay signal over the control. By comparing samples 1, 3, and 4 it is clear that introduction of RS to the assay for signal collection does not adversely affect the assay (samples 1 and 3 are equivalent in intensity), but dilution and treatment of the interfering substances found in plasma returns intensities to that observed in buffer (see samples 1, 3, and 4).

In addition to the quantitative results confirming the ability to remove interfering substances by exchange of binding medium with reading solution and the confirmation that the signal detected corresponds to specific binding, the three components of the complexes were also visualized microscopically to confirm the formation of the complexes. The visualization included imaging of the magnetic beads, as well as detection of the location of signals from the Mag-SML construct and from the DLP. Overlay of the images indicates that the detected complexes include all 3 components, again indicating that the measured signals for the exemplary assays above are due to specific binding complexes.

### Example 2

### TRF Assay for NT-proBNP in a “Wet” Lateral Flow Assay Format

Materials Preparation—To activate the labeling beads, one ml of 0.2 μm Time Resolved Fluorescence latex microparticles (MPs) (Thermo Fisher Fremont, Calif.) at 10% (w/v) solids are combined with 1 ml of 0.5 M MES buffer (pH 6.0), 5.5 ml of deionized H20, 2.3 ml of 50 mg of N-hydroxysuccinimide (ISIHS; Product #24500; Pierce Chemical Company, Rockford, Ill.) per ml deionized H2O and 0.2 ml of 5 mg of 1-ethyl-3-(3-dimethylamino-propyl) carbodiimide hydrochloride (EDC; Product #22980; Pierce Chemical Company) in deionized H2O. The resultant mixture is sonicated on ice for 40 seconds and then allowed to react on a shaker at RT for 30 minutes.
The activated MPs are then centrifuged at 100 C at 10,000×g and washed three times with cold 50 mM MES buffer (pH 6.0) by resuspension and centrifugation cycles. In a typical procedure, the final pellet of MPs is suspended in 3.7 ml of 50 mM MES buffer (pH 6.0) and 2.3 ml of 1.0 mg of mouse anti-(Anti-NTproBNP peptide 63-71 antibody (Hytest., Turku, Finland) in the same buffer are added with mixing, followed by an addition of 5 ml of 0.1 M borate buffer (pH 8.5). The mixture is allowed to incubate at RT for 2 hr and then centrifuged as described above. Subsequently, 10 ml of 50 mM borate buffer (pH 8.5) containing 5 mM ethanolamine (cat. # E-9508; Sigma Chemical Company) is added to the pellet, MPs are suspended, incubated at RT for 30 min, and the suspension is centrifuged as described above.
The remaining hydrophobic sites on MPs are then blocked with FSG blocking solution composed of 0.1% (w/v) of fish skin gelatin (FSG; cat. # G-7765; Sigma Chemical Company) in 50 mM borate buffer (pH 8.5) at RT for 30 min. The MPs blocked with FSG are centrifuged as described above and suspended in 0.2 M EPPS buffer (pH 8.0) containing 0.5% (w/v) of FSG, 0.5% (w/v) of Hammarsten casein (Product #440203H; BDH Laboratory Supplies, Poole, England), 0.5% (v/v) of Tween 20 (cat. # P-1379; Sigma Chemical Company) and 0.01% (w/v) Of NaN3.
To prepare the capture zone membranes, nitrocellulose having a pore size of >5 um is affixed to an XY-plotter table. A Streptavidin capture band is dispensed in a 2.0 mm zone at the distal end of the nitrocellulose membrane using Streptavidin at 2.10 mg/ml. The solution is dispensed with an IVEK Digispense dispensing system. After air drying at 45° C., the membrane is placed into a tray containing the membrane blocking solution comprised of BSA solution at 10 mg/ml for 20 minutes at RT. The membranes are then removed and blotted for 5 minutes. The membranes are air dried at 45° C. for 5 minutes, and then placed at less than 5.0% RH overnight. Processed capture membranes remain at less than 5.0% RH until assembly.
Assay: Ten-fold concentrated Sample Treatment Buffer (STB) for “wet” assays is comprised of 0.5 M EPPS buffer (pH 8.0) supplemented with 6.25% (v/v) of Tween-20 (cat. P 1379; Sigma Chemical Company), 2% (w/v) of BSA and 0.1% (w/v) Of NaN3. For “wet” assays a 14×100 mm strip of the capture zone membrane is affixed centrally on an adhesive opaque strip. The opaque backing is a 23×350 mm strip of 5 mil white mylar laminated with 3M 9502 transfer adhesive. The absorbent-which is a 10×100 mm rectangle of Whatman 31 ET cellulose paper (F075-14, Whatman, Inc., Fairfield, N.J.) is affixed distal to the capture zone pad with ˜0.5 mm overlap. The sample zone pad composed of 7×100 mm cellulose nitrate (Whatman, Inc., Fairfield, N.J.) is then placed next to the capture zone membrane with 0.5 mm overlap.
As illustrated in FIG. 9A, in “wet” assays, 9-10 μl of specimen sample is mixed sequentially in a test tube with 1 μl o>f 10-fold concentrated stock of modified STB, 1 μl of biotinylated antibody of mouse anti-NTrp peptide 13-20 antibody (Hytest., Turku, Finland) and 1 μl of labeling MPs prepared at 0.5% (w/v) solids. Subsequently, the “wet” assay strip assembled as just described is placed into the tube, allowed to develop for 7 min, then removed and the flourescence of the band is measured with a protoype Time resolve Flourence reader (TRF Reader) (Cambridge Consultants LTD, Cambridge, England). Increasing values from the TRF reader indicate increasing flourescence intensity, which corresponds to increasing analyte concentration. FIG. 9B shows a typical Nt-Pro calibration curve generated using our prototype instrument and the modified STB just described. The raw flourescent intensity for each sample test strip is recorded in fluorescence units (FU), and the signal calculated as the area under the fluorescence curve. This flourescence signal is converted to clinical Nt-pro pM using a calibration curve.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the detectable labels used, as well as to the solutions in which the assays are carried out and the apparatus for performing and/or reading the assays. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

What is claimed is:

1. A set of assay reagents, comprising
a first analyte-specific binding reagent comprising a first label;
a second analyte-specific binding reagent comprising a second label, wherein said first and second labels interact to provide a signal indicative of said interaction; and
a complex separation moiety, wherein said complex separation moiety is a part of said first binding reagent or said second binding reagent.

2. The set of assay reagents of claim 1, wherein said separation moiety comprises a magnetic material.

3. The set of assay reagents of claim 1, wherein said separation moiety comprises a surface binding moiety.

4. The set of assay reagents of claim 1, wherein a plurality of members of said set further comprise distinguishable coding moieties.

5. The set of assay reagents of claim 4, wherein said distinguishable coding moieties comprise fluorescent dyes having different fluorescent emission peaks.

6. The set of assay reagents of claim 4, wherein said distinguishable coding moieties comprise fluorescent dyes having different absorption peaks.

7. The set of assay reagents of claim 4, wherein said distinguishable coding moieties comprise dye moieties having different absorption peaks.

8. The set of assay reagents of claim 4, wherein said distinguishable coding moieties comprise different chemiluminescent compounds having different luminescent wavelengths.

9. The set of assay reagents of claim 4, wherein said distinguishable coding moieties comprise enzymes having different enzymatic activities.

10. The set of assay reagents of claim 4, wherein said distinguishable coding moieties comprise particles having distinguishable light scattering properties.

11. The set of assay reagents of claim 1, wherein said first analyte-specific binding reagent comprises a photosensitizer.

12. The set of assay reagents of claim 11, wherein said second analyte-specific binding reagent comprises a chemiluminescent compound that reacts to a product of said photosensitizer.

13. The set of assay reagents of claim 1, wherein said first analyte-specific binding reagent comprises a first fluorescent compound.

14. The set of assay reagents of claim 13, wherein said second analyte-specific binding reagent comprises a second fluorescent compound that accepts energy from said first fluorescent compound.

15. The set of assay reagents of claim 1, wherein said first analyte-specific binding reagent comprises a first enzyme.

16. The set of assay reagents of claim 15, wherein said second analyte-specific binding reagent comprises a second enzyme which uses a product of said first enzyme as a substrate.

17. The set of assay reagents of claim 1, further comprising a signal enhancer.

18. The set of assay reagents of claim 1, further comprising a reading solution.

19. An assay complex, comprising
a first analyte-specific binding moiety comprising a first label;
a second analyte-specific binding moiety comprising a second label, wherein said first and second moieties interact to provide a signal indicative of said interaction;
an analyte bound to said first moiety and said second moiety; and'
a separation moiety, wherein said separation moiety is a part of said first binding moiety or said second binding moiety.

20. An assay kit comprising
a first analyte-specific binding reagent comprising a first label;
a second analyte-specific binding reagent comprising a second label, wherein said first and second labels interact to provide a signal indicative of said interaction;
a separation moiety, wherein said separation moiety is attached to said first reagent or said second reagent; and
instructions for performing an analyte assay using said first and second reagents.

21. The kit of claim 20, further comprising a reading solution.

22. A single-use assay device comprising
a sample reservoir;
a first analyte-specific binding reagent comprising a first label;
a second analyte-specific binding reagent comprising a second label, wherein said first and second labels interact to provide a signal indicative of said interaction and wherein said first or second binding reagent comprises a separation moiety;
a signal detection chamber in fluid connection with said sample reservoir; and
a signal detection solution reservoir;
wherein said first and second reagents are in fluid connection with said sample reservoir and said signal detection solution is in fluid connection wth said signal detection chamber.

23. The assay device of claim 22, wherein said device is a microfluidic device.

24. The assay device of claim 22, wherein said device comprises a plurality of coding labels providing distinguishably different detectable coding signals, wherein co-occurrence of a particular coding signal with a signal from the interaction of said first and second labels is indicative of the binding of a particular analyte.

25. An assay reading device comprising
a magnetic controller configured to apply a magnetic field to an assay device positioned for reading in said assay reading device; and
at least one signal detector configured to detect signals indicative of analyte binding in said assay device for at least two different analytes.

26. The assay reading device of claim 25, wherein said signal detector comprises fluorescence detectors.

27. The assay device of claim 25, wherein said assay device is a home use device.

28. The assay device of claim 25, wherein said assay device is a point-of-care device.

29. A method for analyzing one or more analytes in a solution, comprising
forming an assay complex in a binding medium
displacing said binding medium with a reading solution
detecting a signal from said assay complex.

30. The method of claim 29, wherein said displacing is performed as a single-step displacement.

31. The method of claim 29, wherein said displacing is a low volume displacement.

32. The method of claim 29, wherein said one or more analytes is at least two analytes.

33. The method of claim 29, wherein said one or more analytes is at least 4 analytes.

34. The method of claim 29, wherein said assay complex comprises a signal modulation label and a detection label.

35. A method for enhancing detection of one or more analytes in a solution, comprising
retarding an analyte-specific sandwich binding complex in a flow device;
displacing binding medium surrounding said complex by flow of a liquid reading solution; and
detecting a signal indicative of the presence of said analyte from said binding complex in said reading solution, wherein the specific detection of said analyte is enhanced compared to detection in said binding medium.

36. The method of claim 35, wherein said binding medium is blood diluted no more than 20 percent.

37. The method of claim 35, wherein said binding medium is serum diluted no more than 20 percent.

38. The method of claim 35, wherein said binding medium is a crude cell extract diluted no more than 20 percent.

39. The method of claim 35, wherein said displacing is performed in a single step.

40. The method of claim 35, wherein said displacing is performed using no more than 50 microliters of reading solution.

41. The method of claim 35, wherein said detecting comprises detecting a plurality of signals indicative of the presence of a plurality of different analytes.

42. The method of claim 41, wherein said plurality of different analytes comprises at least 3 different analytes.

43. A method for detecting the presence or amount or both of an analyte in a solution, comprising
binding an analyte-specific binding construct with an analyte in a solution; and
detecting a signal from a full-coat label linked with said analyte-specific binding construct, wherein detection of said signal is indicative of the presence or amount or both of said analyte in said solution.

44. The method of claim 43, wherein said label is a layered label.

45. The method of claim 43, wherein said label is a fully linked coating label.

46. The method of claim 43, wherein said solution is blood.

47. The method of claim 43, wherein said solution is plasma.

48. The method of claim 43, wherein said solution is applied to a lateral flow assay device and said detecting is performed on said device.

49. The method of claim 44, wherein said layered label comprises a solid phase core bearing a plurality of detectable signal moieties and at least two linked hydrophilic polymer layers coating said core.

50. The method of claim 44, wherein said layered label comprises at least two linked hydrophilic polymer layers comprising a plurality of detectable signal moieties embedded in said layers.

51. The method of claim 44, wherein said layered label comprises a plurality of linked hydrophilic polymer layers without a solid phase core.

52. The method of claim 44, wherein said layered label comprises a solid phase core and at least two hydrophilic polymer coating layers, wherein said layered label has substantially less non-specific protein binding for proteins in undiluted human plasma than a coated label having the same solid phase core and a single coating of the same hydrophilic polymer as forms the outermost coating layer of said coated polymer.

53. The method of claim 45, wherein said fully linked coating label comprises a solid phase core particle and a highly linked protein coating.

54. The method of claim 53, wherein said highly linked protein coating is linked to said particle through naturally occurring amine groups.

55. The method of claim 54, where said highly linked protein coating comprises reduced disulfide bonds.

56. The method of claim 55, wherein at least one analyte specific binding moiety is linked to said protein through —SH groups created by reducing said disulfide bonds.

57. The method of claim 53, wherein said label is a colorimetric label.

58. The method of claim 53, wherein said label is a fluorescent label.

59. The method of claim 53, wherein said label is a luminescent label.

60. The method of claim 53, wherein said label is a radioactive label.

61. An assay kit, comprising
a measured quantity of a first analyte specific binding construct; and
at least one lateral flow assay device, wherein said first analyte specific binding construct is separate from said assay device.

62. The assay kit of claim 61, wherein said assay device is configured to perform a wet assay.

63. The assay kit of claim 62, wherein said assay device is configured to perform field mixing of sample and said first analyte specific binding construct in said device.

64. The assay kit of claim 62, wherein said assay device is configured to assay a sample of 10 microliters or less.

65. The assay kit of claim 62, wherein a controlled volume is extracted from a raw sample in said assay device.

66. The assay kit of claim 62, wherein said mixing is performed using electrowetting effects.

67. A layered particulate label, comprising
a plurality of polymer layers, wherein at least the outermost of said layers provides low non-specific protein binding; and
a plurality of detectable label moieties.

68. The label of claim 67, comprising two polymer layers.

69. The label of claim 67, comprising at least three polymer layers.

70. The label of claim 67, wherein one or more outer polymer layers are permeable to water.

71. The label of claim 67, wherein said label comprises a solid phase core.

72. The label of claim 71, wherein said solid phase core comprises a plurality of detectable signal moieties.

73. The label of claim 71, wherein said polymer layers comprise a plurality of detectable signal moieties.

74. The label of claim 73, wherein a plurality of detectable signal moieties are embedded in said polymer layers.

75. The label of claim 67, wherein said label lacks a solid phase core.

76. The label of claim 75, wherein a plurality of detectable signal moieties are embedded in said polymer layers.

77. The label of claim 67, further comprising a plurality of binding moieties which bind with an analyte-specific binding moiety.

78. The label of claim 67, wherein said label is linked with at least one analyte-specific binding moiety.

79. The label of claim 78, wherein said label is linked with at least one analyte.

80. The label of claim 79, wherein said label is immobilized in a signal detection zone of a lateral flow assay device by linkage with immobilized analyte.

81. A method for detecting the presence or amount or both of an analyte in a solution, comprising
depositing a fluid sample in a sample deposition zone of a lateral-flow assay device comprising a solid phase strip;
depositing a specific binding reagent in a reagent deposition zone of said assay device, wherein said sample deposition zone and said reagent deposition zone may be the same or different;
mixing said sample and said specific binding reagent using a field mixer to form a sample-reagent mixture, whereby said reagent specifically binds with analyte if any in said sample;
migrating said sample-reagent mixture along said device to a signal detection zone; and
detecting signal in said signal detection zone as an indication of the present or amount or both of analyte in said sample.

82. The method of claim 81, further comprising preparing said sample within said device.

83. The method of claim 82, wherein preparing said sample comprises separating liquid from cells.

84. The method of claim 83, wherein said cells are blood cells.

85. The method of claim 81, wherein said specific binding reagent is applied with said sample.

86. The method of claim 81, wherein said specific binding reagent is applied separately from said sample.

87. The method of claim 81, wherein said specific binding reagent is dried onto a portion of said strip upstream of said signal detection zone.

88. The method of claim 81, wherein said device further comprises an electrowetting fluid manipulation electrode array.

89. The method of claim 88, wherein said electrode array is used to mix a volume of said sample.

90. The method of claim 89, wherein said electrode array is used to move a volume of said sample into contact with said solid phase strip.

91. A lateral flow assay device, comprising
a sample deposition zone;
a reagent deposition zone;
a field mixing zone;
a solid phase strip in contact with said field mixing zone and comprising a signal detection zone; and
a fluid collection zone in contact with said solid phase strip distal to said field mixing zone and said signal detection zone.

92. The device of claim 91, wherein said field mixing zone comprises an electrowetting fluid manipulation electrode array.

93. The device of claim 92, wherein said electrode array is configured to also move a droplet of fluid.

94. The device of claim 91, further comprising a filter or binding moiety or both selected to retain cells present in a sample.

95. The device of claim 91, wherein said solid phase strip comprises nitrocellulose.

96. The device of claim 91, wherein said signal detection zone comprises immobilized analyte-specific binding moieties.

97. The device of claim 91, wherein said fluid collection zone comprise an absorbent material.

98. The device of claim 91, wherein said device provides useful results when used with a liquid sample of 10 microliters or less.

99. The device of claim 91, wherein a sample volume of no more than 5 microliters is passed over said solid phase strip.
